(19) United States
(12) Patent Application Publication
(10) Pub. No.: US 2021/0128414 A1
(43) Pub. Date: May 6, 2021

(54) SELF-ASSEMBLED NANOSTRUCTURES AND COMPOSITE MATERIALS USABLE IN DENTAL APPLICATIONS CONTAINING SAME

(71) Applicant: Ramot at Tel-Aviv University Ltd., Tel-Aviv (IL)

(72) Inventors: Lihi ADLER-ABRAMOVICH, Tel Aviv (IL); Lee SCHNAIDER, Tel-Aviv (IL); Shlomo MATALON, Tel-Aviv (IL); Tamar BROSH, Tel-Aviv (IL); Raphael PILO, Tel-Aviv (IL); Ehud GAZIT, Tel-Aviv (IL)

(73) Assignee: Ramot at Tel-Aviv University Ltd., Tel-Aviv (IL)

(21) Appl. No.: 17/145,465

(22) Filed: Jan. 11, 2021

Related U.S. Application Data
(63) Continuation of application No. PCT/IL2019/050788, filed on Jul. 12, 2019.
(60) Provisional application No. 62/696,879, filed on Jul. 12, 2018.

(51) Int. Cl. A61K 6/52; A61K 6/20

(52) U.S. Cl. CPC A61K 6/52; B82Y 5/00; A61K 6/20

(57) ABSTRACT

A composition containing a plurality of self-assembled nanostructures formed of a plurality of aromatic molecules which include an aromatic amino acid, and which exhibits an antibacterial activity is provided. The composition can be a dental composition which further comprises a dental formulation such as a curable dental formulation, for forming dental composite materials such as dental restorative composite materials. Processes of preparing the composition and uses thereof are also provided.

### RELATED APPLICATIONS

This application is a US Continuation of PCT Patent Application No. PCT/IL2019/050788 having international filing date of Jul. 12, 2019 which claims the benefit of priority under 35 USC § 119(e) of U.S. Provisional Patent Application No. 62/696,879 filed on Jul. 12, 2018. The contents of the above applications are all incorporated by reference as if fully set forth herein in their entirety.

### FIELD AND BACKGROUND OF THE INVENTION

The present invention, in some embodiments thereof, relates to dentistry and, more particularly, but not exclusively, to compositions featuring an anti-bacterial and/or anti-biofilm formation activity which are usable in dental applications such as formation of dental restorative composite materials, which feature anti-bacterial activity, and to composite materials prepared therefrom. The present invention, in some embodiments thereof, further relates to compositions featuring an anti-bacterial and/or anti-biofilm formation activity and to uses thereof in inhibiting, reducing and/or preventing a bacterial load and/or in inhibiting, reducing and/or retarding biofilm formation in and/or on a substrate.

Dental infections such as dental caries (tooth decay) and periodontal diseases are pressing global oral health burdens affecting 60-90% of school-children and the vast majority of adults. Dental caries is one of the most prevalent and costly oral diseases caused by the acidification of tooth enamel and dentin by virulent bacterial species, such as Streptococcus mutans (S. mutans) and other bacteria. These bacteria accumulate on the tooth surface and ultimately dissolve the hard tissues of the teeth.

Recurrent caries, also known as secondary tooth decay, at the margins of dental restorations, is the result of acid production by caries-causing bacteria that reside in the restoration-tooth interface. This malady is a major causative factor for dental restorative material failure, and has been estimated to affect over 100 million patients a year, at an estimated cost of over 30 billion dollars. In addition to acid production, enzymes produced by caries-causing bacteria degrade the materials and the resultant marginal leakage at the restoration-tooth interface contributes to the formation and progression of recurrent caries; emphasizing the need for the fabrication of resin composites containing constituents that also display bacterial inhibitory activity. Nowadays, a number of substances with effective antimicrobial activity that inhibit biofilm formation in the oral cavity include substances such as chlorhexidine, delmopinol, and phenolic compounds. For an exemplary review of current treatment methodologies see, for example, Krzyściak et al., Eur J Clin Microbial Infect Dis (2014) 33:499-515. Some of these substances are known as involving side effects such as vomiting, diarrhea, addiction, or teeth discoloration.

Researches have also focused on developing antibacterial dental materials such as, for example, resin-based pit-and-fissure restorative composite materials, modified by the addition of soluble antimicrobials. The incorporated antibacterial moieties can either be released as a soluble agent or remain in the resin in a stationary phase. The most prominent agents introduced include classical antibiotics, fluoride, chlorhexidine, antibacterial nanomaterials and carriers, silver-based moieties, iodine, zinc, and quaternary ammonium compounds. However, the gradual release of soluble agents from the bulk resin has an adverse influence on the mechanical properties as the leaching may result in a porous and weak resin. Furthermore, the antibacterial activity in these cases is time-limited and the released compounds may display cytotoxic activity toward the adjacent human tissues. These shortcomings are amplified when taking into account the relatively high w/w % loading dose needed to effectively inhibit bacterial growth and reduce bacterial viability, which can often reach tens of percentages. Reference is made, for example, to Bourbia et al. J. Dent. Res. 2013, 92, 989-994; Cocco et al. Current Status and Further Prospects. Dent. Mater. 2015, 31, 1345-1362; Imazato et al. Dent. Mater. 2014, 30, 97-104; Melo et al. Trends Biotechnol. 2013, 459-467; Beyth et al. React. Funct. Polym. 2014, 75, 81-88; and Beyth et al., J. Antimicrobial Nanoparticles in Restorative Composites. In Emerging Nanotechnologies in Dentistry, 2nd ed.; William Andrew Publishers, 2018; pp 41-58.

Peptide-based antimicrobial materials have also been developed utilizing relatively long peptides which are introduced as additives within dental resin composite restoratives or are able to directly bind hydroxyapatite. The lengths of these peptides cause these agents to be costly and it is hard to achieve a high degree of purity for such agents. Nanoparticles have also been utilized in order to develop resin composite restoratives with antimicrobial properties. These nanoparticles incorporate a wide variety of materials such as metals, quaternary ammonium methacrylates, amorphous calcium phosphate and polyethylenimines.

Self-assembled, biocompatible, peptide-based hydrogels have been widely explored in recent years, particularly for biotechnological and medical applications [Fleming, S. & Ulijn, R. V. Chem. Soc. Rev.43, 8150-8177, (2014); Fichman, G. & Gazit, Acta Biomater.10, 1671-1682, (2014)]. These self-assembled hydrogels have been found to form a support scaffold for the growth of cells and are being used in the field of regenerative medicine [Ellis-Behnke, R. G. et al. Proc. Nat. Acad. Sci. U.S.A.103, 5054-5059, (2006)]. The self-assembled ultra-short peptide building blocks are easy to fabricate and can be simply chemically and biologically decorated [Mahler et al. Adv. Mater.18, 1365-1370, (2006); Jayawarna, V. et al. Adv. Mater.18, 611-614, (2006)].

WO 2004/052773 and WO 2004/060791 disclose self-assembled peptide tubular nanostructures made of short aromatic peptides, and uses thereof.

WO2007/043048 and Reches and Gazit [Isr. J. Chem. 2005; 45: 363-371] disclose the assembly of tubular and fibrillar (amyloid-like) structures from a plurality of non-charged, end-capping modified aromatic peptides.0

Adler-Abramovich et al. [J. Pept. Sci. 2008; 14: 217-223] describe that two types of nanostructures—nanotubes and nanospheres, are obtained by the self-assembly of the aromatic dipeptide Phe-Phe, while using different end-capping moieties.

Ample studies have focused on Fmoc-modified oligopeptides and their ability to form hydrogels. See, for example, Burch, R. M. et al. Proc. Nat. Acad. Sci. U.S.A.88, 355-359, (1991). An example of Fmoc-based hydrogels is the Fmoc-μF peptide that efficiently assembles into fibrous hydrogels under physiological conditions [Jayawarna, V. et al. Adv. Mater.18, 611-614, (2006); Mahler et al., 2006, supra; and WO 2007/043048]. The properties of the fibrous hydrogels have been characterized and used for various applications [Adler-Abramovich, L. & Gazit, E. Chem. Soc. Rev.43, 6881-6893, (2014)].

The single amino acid phenylalanine was shown to form ordered structures [Adler-Abramovich, L. et al. Nat. Chem. Biol. 8, 701-706, (2012)], and Fmoc-modified aromatic single amino acids analogues, Fmoc-Phe and Fmoc-Tyr were also shown to form ordered fibrillar assemblies [Draper, E. R. et al. CrystEngComm17, 8047-8057, (2015)].

Fmoc-modified aromatic non-coded single amino acids have also been investigated as hydrogelators. See, for example, Fichman et al. CrystEngComm 17, 8105-8112, (2015); Orbach, R. et al. Biomacromolecules 10, 2646-2651, (2009); and Ryan et al. Soft Matter 6, 3220-3231, (2010).

The fluorinated peptide derivative of Fmoc-Phe, Fmoc-pentafluorophenylalanine (Fmoc-F5-Phe), has been reported to rapidly self-assemble into ordered structures [Ryan et al. Soft Matter 6, 3220-3231, (2010)].

In spite of their advantages, the physical properties of short peptide-based and amino acid-based hydrogels are limited due to the chemical nature of the chosen building blocks, making the modulation of the physical properties highly challenging in each case.

Co-assembly of two building blocks into one ordered structure has been shown to provide a new material exhibiting enhanced properties. It has been shown that the co-assembly of short peptide building blocks can produce complex architectures such as “beads on a string”, hydrogels and tubes. See, for example, Orbach et al. Langmuir 2012, 28, 2015-2022; Carny et al. Nano Lett. 2006, 6, 1594-7.

Sedman et al. [J. of Microscopy, 2013, pp. 1-8] teach nano- and micro-scale fibrillar and tubular structures formed by mixing two aromatic dipeptides, Phe-Phe and D-Nal-Nal, and describe that the mechanical properties of the structures depend on the percentage of each peptide in the mixture.

Yuran et al. [ACS Nano, 2012, 6 (11), pp 9559-9566] describe the formation of complex peptide-based structures by the co-assembly of Phe-Phe-OH and Boc-Phe-Phe-OH, into a construction of beaded strings, where spherical assemblies are connected by elongated elements.

Maity et al. [J. Mater. Chem. B, 2014, 2, 2583-2591] describe the co-assembly of two aromatic dipeptides, diphenylalanine and Fmoc-L-DOPA(acetonated)-D-Phe-OMe, into different spherical structures that are similar in morphology to either red or white blood cells.

Maity et al. Chem. Commun. 50, 11154-11157, (2014) have utilized the carbon-fluorine bond of the fluorinated aromatic ring of Pentafluoro-phenylalanine as an antifouling motif incorporated into a tripeptide, Dopa-di-Pentafluoro-phenylalanine, that self-assembles to form a functional coating that resists fouling.

U.S. Patent Application Publication No. 2016-0326215 describes self-assembled hybrid materials formed of two types of aromatic dipeptides, which differ from one another by the type and/or presence of their end-capping moiety.

Additional self-assembled hybrid materials include, for example, synthetic triskelion peptide, which self-assembles into spherical structures, co-assembled with diphenylalanine fibrils [Ghosh, S. & Verma, S. Chem. Eur. J. 14, 1415-1419, (2008)]; co-assembly of Fmoc-F5-Phe with PEG-functionalized monomers was described [Ryan, D. M., Anderson, S. B. & Nilsson, B. L. Soft Matter 6, 3220-3231, (2010)]; and co-assembly of Fmoc-FF and Fmoc-FG was also described [Orbach, R. et al. Langmuir 28, 2015-2022, (2012)].

Schnaider, L. et al. Nat. Commun. 8, 1365 (2017) describe that nano-assemblies formed by the diphenylalanine building block have substantial antibacterial and membrane interacting activity.

Additional background art includes Mandal et al., Chem. Commun. 48, 1814-1816, (2012); Li, J. et al. J. Am. Chem. Soc.135, 542-545, (2013); Wang et al. Nature 463, 339-343, (2010); Jayawarna, et al. Acta Biomater.5, 934-943, (2009); Cheng et al. Langmuir 26, 4990-4998, (2010); Dudukovic, N. A. & Zukoski, C. F. Langmuir 30, 4493-4500, (2014); Van Loveren, C. Caries Res.35, 65-70, (2001); Martin et al. Chem. Commun.50, 15541-15544, (2014); Shekhter-Zahavi, T. et al. ChemNanoMat3, 27, (2017); Sedman et al., J Microsc. 2013 March; 249(3): 165-172; Adler-Abramovich, L. et al. ACS Nano, (2016); Timothy J. Mitchel, Nature Reviews Microbiology, Volume 1, December 2003. pp. 227-230; Walter J. Loesche, Microbiological Reviews, December 1986, p. 353-380; Hamada and Slade, Microbiological Reviews, June 1980, p. 331-384; and Schnaider et al., ACS Appl. Mater. Interfaces 2019, 11, 21334-21342.

### SUMMARY OF THE INVENTION

Embodiments of the present invention relate to compositions usable in dental applications such as formation of dental restorative composite materials and other materials that are usable in treating or preventing an infection of biofilm formation in the oral cavity. The compositions comprise self-assembled nanostructures that exhibit an antibacterial and/or anti-biofilm formation activity, and feature, in addition, mechanical and/or optical properties that meet the requirements of their intended use.

According to an aspect of some embodiments of the present invention there is provided a composition comprising a dental formulation and at least one self-assembled nanostructure incorporated in the dental formulation, the nanostructure being formed of self-assembled plurality of aromatic molecules, wherein each of the aromatic molecules comprises an aromatic amino acid. This composition is also referred to herein as a dental composition.

According to some of any of the embodiments described herein, the dental formulation is a curable formulation which comprises at least one polymeric precursor.

According to some of any of the embodiments described herein, the curable dental formulation is configured for forming a polymeric matrix for a dental application.

According to some of any of the embodiments described herein, the curable dental formulation is configured for forming a dental restorative material.

According to some of any of the embodiments described herein, in at least a portion of the plurality of aromatic molecules, each of the aromatic molecules comprises an aromatic amino acid having an end-capping moiety attached thereto.

According to some of any of the embodiments described herein, the end-capping moiety is an aromatic end-capping moiety.

According to some of any of the embodiments described herein, the end-capping moiety is attached to the alpha-amine of the aromatic amino acid.

According to some of any of the embodiments described herein, in at least a portion of the plurality of aromatic molecules, each of the aromatic molecules comprises a peptide of from 2 to 6 amino acid residues, at least one of the amino acid residues being the aromatic amino acid.

According to some of any of the embodiments described herein, the peptide is a di-peptide. According to some of any of the embodiments described herein, the peptide is an end-capping modified peptide.

According to some of any of the embodiments described herein, the end-capping modified peptide is an N-terminus modified peptide.

According to some of any of the embodiments described herein, the end-capping modified peptide comprises an aromatic end-capping moiety.

According to some of any of the embodiments described herein, the aromatic end-capping moiety is Fmoc.

According to some of any of the embodiments described herein, the aromatic amino acid is phenylalanine.

According to some of any of the embodiments described herein, in at least a portion of the aromatic molecules, the aromatic amino acid is a halogenated aromatic amino acid.

According to some of any of the embodiments described herein, the halogenated aromatic amino acid comprises in its side chain an aromatic moiety substituted by 1, 2, 3, 4, 5 or more halogen substituents.

According to some of any of the embodiments described herein, the halogenated aromatic amino acid is a fluorinated aromatic amino acid.

According to some of any of the embodiments described herein, the halogenated aromatic amino acid is a halogenated phenylalanine.

According to some of any of the embodiments described herein, the halogenated aromatic amino acid is pentafluoro-phenylalanine.

According to some of any of the embodiments described herein, the plurality of aromatic molecules comprises a plurality of Fmoc-pentafluoro-phenylalanine.

According to some of any of the embodiments described herein, the plurality of aromatic molecules comprises a plurality of Fmoc-phenylalanine.

According to some of any of the embodiments described herein, the at least one nanostructure exhibits an anti-bacterial activity and/or an anti-biofouling activity.

According to some of any of the embodiments described herein, a weight ratio of the at least one nanostructure and the polymeric precursor mixture ranges from 1:1000 to 1:10, or from 1:100 to 1:10, or from 1:100 to 1:20, or from 1:100 to 1:50.

According to some of any of the embodiments described herein, the dental composition as described herein is for use in treating or preventing a dental and/or periodontal infection.

According to some of any of the embodiments described herein, the dental composition as described herein is for use in forming a dental restorative material.

According to some of any of the embodiments described herein, the dental composition as described herein is for use in forming a medical device or material for dental, periodontal or orthodontic application.

According to some of any of the embodiments described herein, the medical device or material is for treating a dental, periodontal or orthodontic condition in which treating or preventing a bacterial infection and/or reducing, inhibiting or retarding biofilm formation is beneficial.

According to some of any of the embodiments described herein, the dental composition as described herein is for use in treating a dental, periodontal or orthodontic condition in which treating or preventing a bacterial infection and/or reducing, inhibiting or retarding biofilm formation is beneficial.

According to an aspect of some embodiments of the present invention there is provided a composite material comprising a polymeric matrix usable in a dental, periodontal or orthodontic application and at least one self-assembled nanostructure incorporated in and/or on the polymeric matrix, the composite material being prepared upon subjecting the dental composition as described herein in any of the respective embodiments to conditions for effecting curing of the curable formulation.

According to an aspect of some embodiments of the present invention there is provided a composite material comprising a polymeric matrix usable in a dental application and at least one self-assembled nanostructure incorporated in and/or on the polymeric matrix, wherein: the polymeric matrix is usable in a dental, periodontal or orthodontic application; and the at least one nanostructure comprises a nanostructure formed of a plurality of aromatic molecules, each of the aromatic molecules comprising an aromatic amino acid.

According to some of any of the embodiments described herein, the at least one nanostructure is as described in any of the respective embodiments.

According to some of any of the embodiments described herein, the polymeric matrix is obtainable upon polymerizing a polymeric precursor as described herein.

According to some of any of the embodiments described herein, the polymeric matrix is obtainable upon exposing a curable dental formulation as described herein to a condition that induces or promotes polymerization of the polymeric precursor.

According to some of any of the embodiments described herein, a toughness of the composite material differs from a toughness of the same polymeric matrix without the at least one nanostructure by no more than 15%.

According to some of any of the embodiments described herein, a Tensile Strength of the composite material differs from a Tensile Strength of the same polymeric matrix without the at least one nanostructure by no more than 15%.

According to some of any of the embodiments described herein, a stiffness of the composite material differs from a stiffness of the same polymeric matrix without the at least one nanostructure by no more than 15%.

According to some of any of the embodiments described herein, a color of the composite material differs from a color of the same polymeric matrix without the at least one nanostructure by no more than 15%, when measured using Spectroshade Micro-MHT dental spectrophotometer normalized to the Vita classical color guide.

According to some of any of the embodiments described herein, no more than 5% by weight of the at least one nanostructure are released from the composite material upon contacting saliva for 24 hours.

According to some of any of the embodiments described herein, the composite material is characterized as featuring an antimicrobial activity.

According to some of any of the embodiments described herein, the composite material is characterized as non-toxic to eukaryotic cells.

According to some of any of the embodiments described herein, the composite material is for use in treating a dental, periodontal or orthodontic condition in which treating or preventing a bacterial infection and/or reducing, inhibiting or retarding biofilm formation is beneficial.

According to some of any of the embodiments described herein, composite material as described herein in any of the respective embodiments is a dental composite material, for example, a dental restorative material.

According to an aspect of some embodiments of the present invention there is provided a process of preparing the dental composition as described herein in any of the respective embodiments, the process comprising: mixing the at least one nanostructure and the polymeric precursor formulation, the mixing comprising repetitively subjecting a mixture of the at least one nanostructure and the polymeric precursor formulation to manual mixing, centrifugation and/or sonication.

According to some of any of the embodiments described herein, the process further comprises, prior to the mixing, forming the at least one nanostructure, the forming comprising diluting a solution comprising the aromatic molecules and an organic solvent with an aqueous solution.

According to an aspect of some embodiments of the present invention there is provided a method of treating or preventing a dental and/or periodontal infection, the method comprising contacting an infected area in the oral cavity of a subject in need thereof with a composition or with the composite material as described herein in any of the respective embodiments. According to an aspect of some embodiments of the present invention there is provided a method of treating a dental, periodontal or orthodontic condition in which treating or preventing a bacterial infection and/or reducing, inhibiting or retarding biofilm formation is beneficial in a subject in need thereof, the method comprising contacting an organ or a tissue in the oral cavity of the subject with a dental composition of or with the dental composite material as described herein in any of the respective embodiments.

According to an aspect of some embodiments of the present invention there is provided a composition comprising at least one nanostructure formed of self-assembled plurality of aromatic molecules, wherein each of the aromatic molecules comprises a halogenated aromatic amino acid, the composition being for use in inhibiting, reducing or retarding a formation of a bacterial load in and/or a substrate. Such a composition is also referred to herein as an antibacterial or an ABF composition.

According to some of any of the embodiments described herein, the composition further a pharmaceutically acceptable carrier, and is referred to herein as a pharmaceutical composition.

According to some of any of the embodiments described herein, the composition further comprises a curable formulation, wherein the at least one nanostructure is incorporated in the curable formulation. The curable formulation can comprise a polymeric precursor, for example, as described herein.

According to an aspect of some embodiments of the present invention there is provided an article-of-manufacture comprising a polymeric matrix and the antibacterial composition as described herein incorporated in and/or the polymeric matrix.

According to an aspect of some embodiments of the present invention there is provided a method of inhibiting, reducing or retarding a formation of a bacterial load in and/or a substrate, the method comprising contacting the substrate with the antibacterial composition as described herein in any of the respective embodiments.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D present micrographs obtained by Scanning Electron Microscopy (SEM) (FIGS. 1A-C) and by Transmission Electron Microscopy (FIG. 1D) for exemplary lyophilized self-assembled nanostructures according to some of the present embodiments.

FIGS. 1E-F present comparative plots showing the bacterial growth inhibition kinetics by various concentrations of Fmoc-F5-Phe self-assembled nanostructures as evaluated by turbidity analysis via absorbance readings at 650 nm (FIG. 1E) and the antibacterial effect of these nanostructures as determined using the Live/Dead backlight bacterial viability kit. Green fluorescence of the Syto9 probe indicates bacterial cells with an intact membrane, while red fluorescence of Propidium Iodide (PI) indicates dead bacterial cells.

FIGS. 1G-H present comparative plots showing the bacterial growth inhibition kinetics following addition 2 mM Fmoc-F5-Phe self-assembled nanostructures to mid-log-phase bacteria, as evaluated by turbidity analysis via absorbance readings at 650 nm (FIG. 1G) and the antibacterial effect of these nanostructures following 4 hours incubation with mid-log-phase bacteria as determined using the Live/Dead backlight bacterial viability kit. Green fluorescence of the Syto9 probe indicates bacterial cells with an intact membrane, while red fluorescence of Propidium Iodide (PI) indicates dead bacterial cells.

FIG. 11 presents micrographs showing the effect of Fmoc-F5-Phe self-assembled nanostructures on bacterial morphology. Micrographs were obtained using a high-resolution scanning electron microscope. The scale bar is 1 μm.

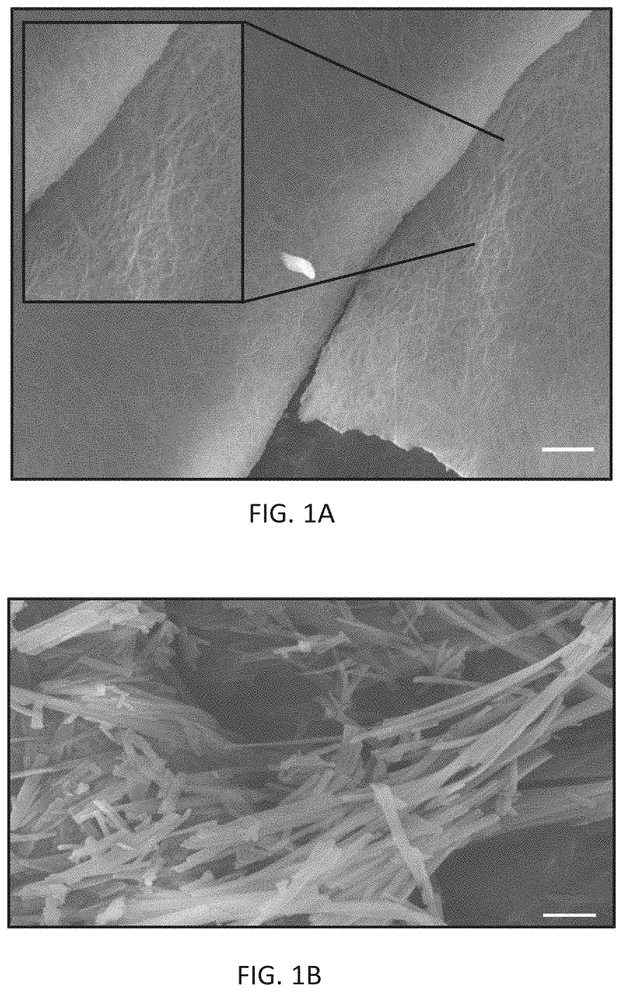

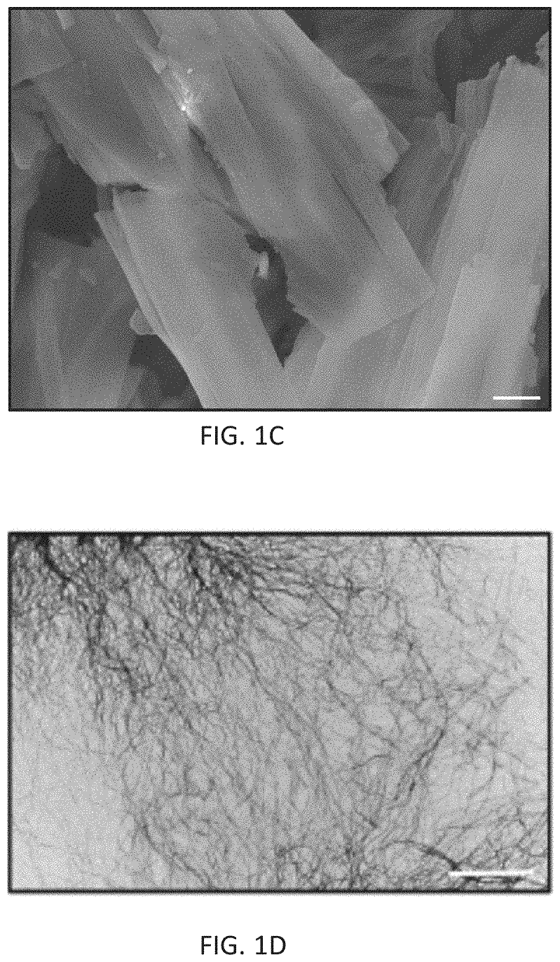

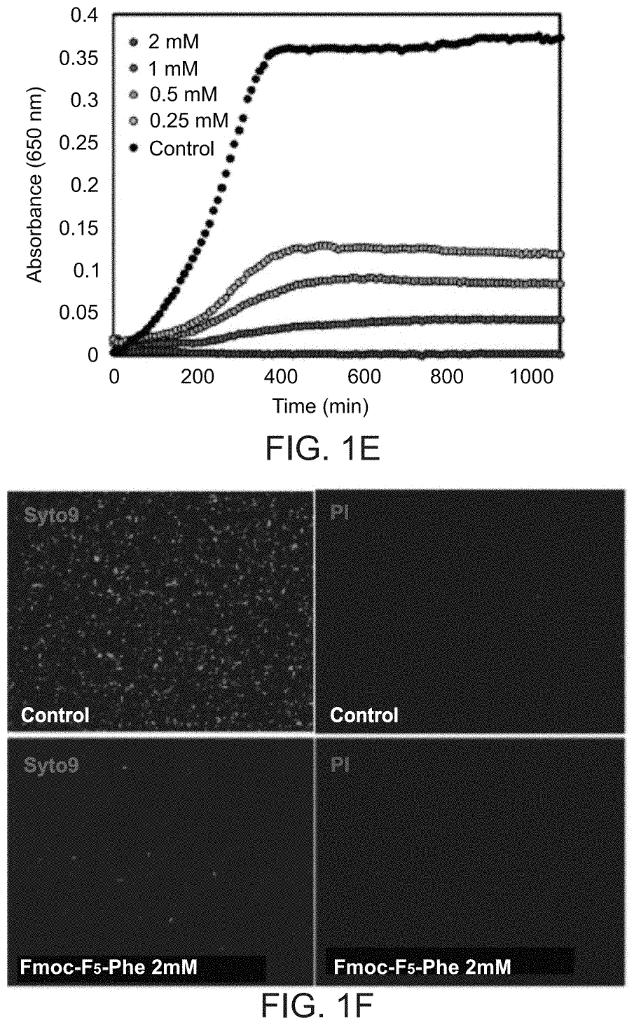

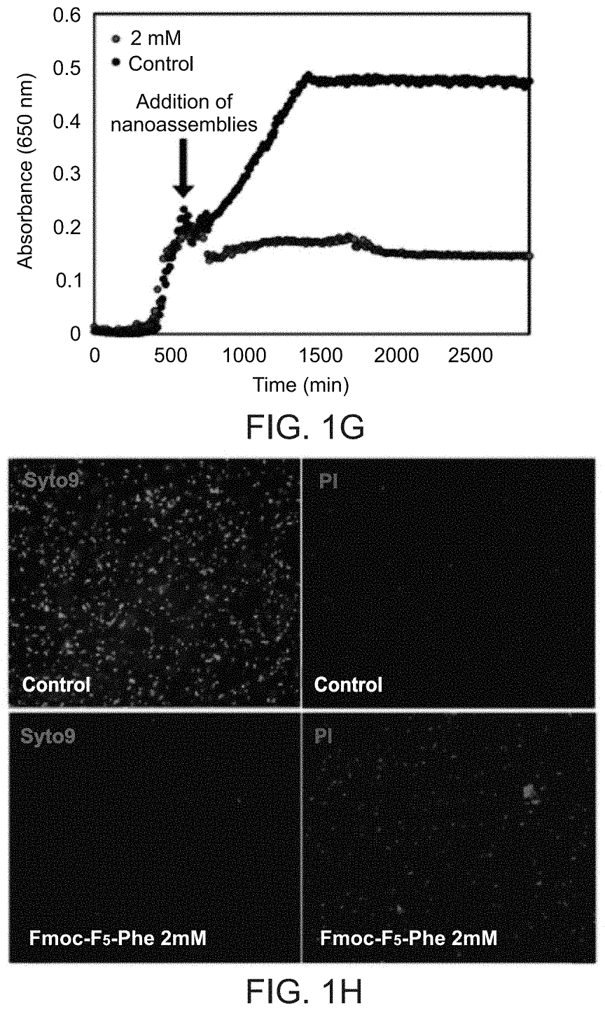

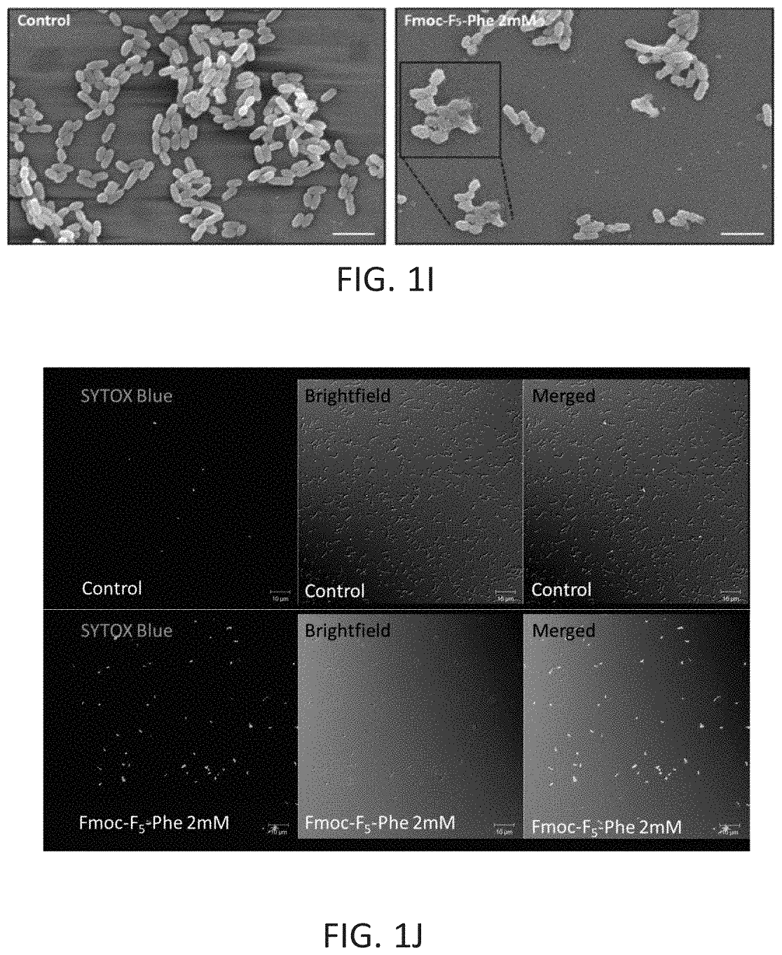

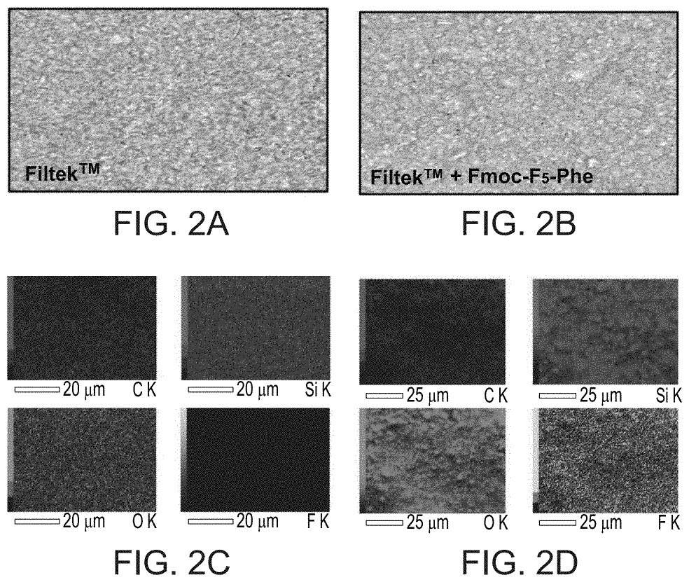

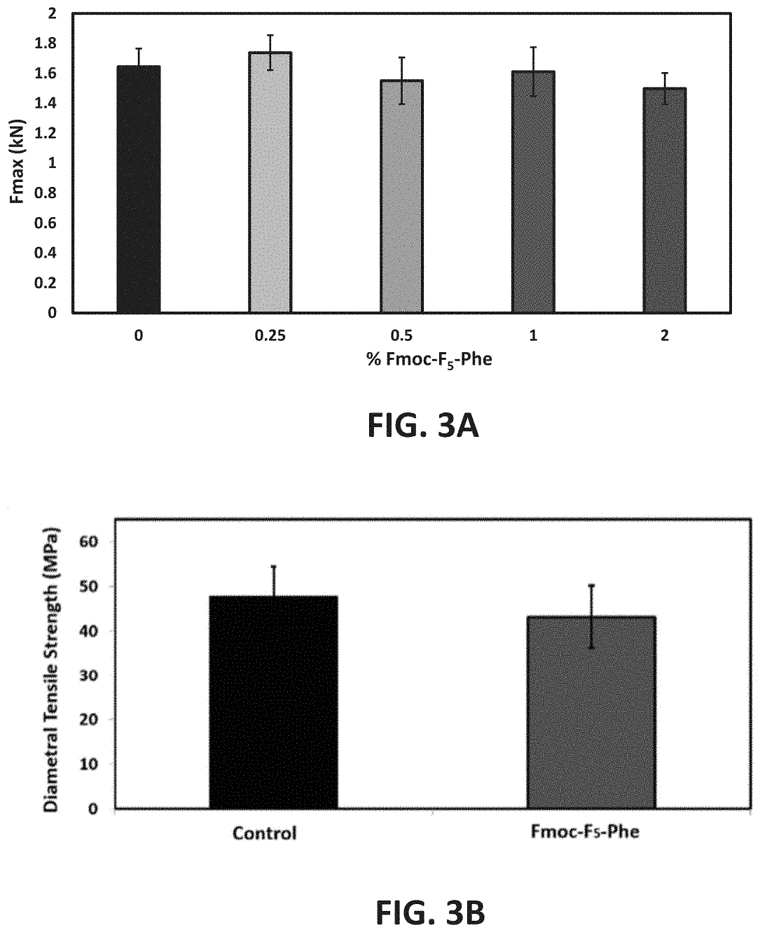

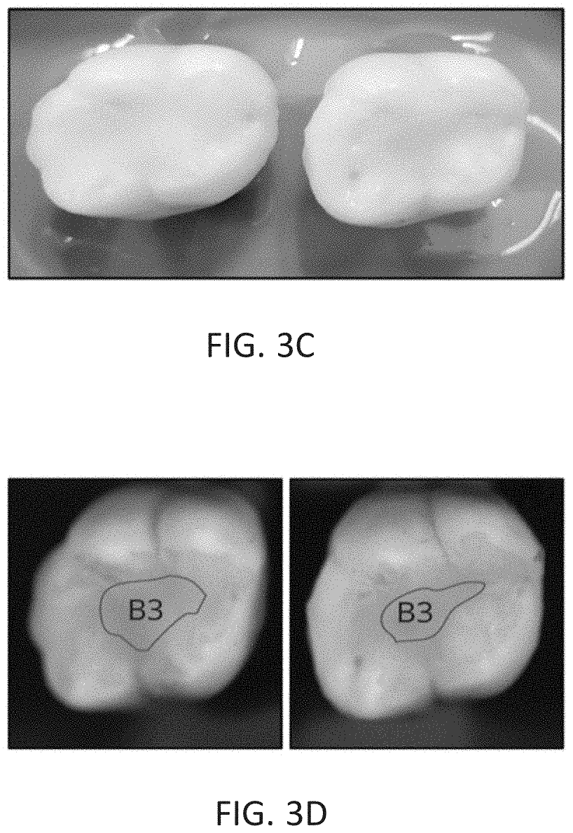

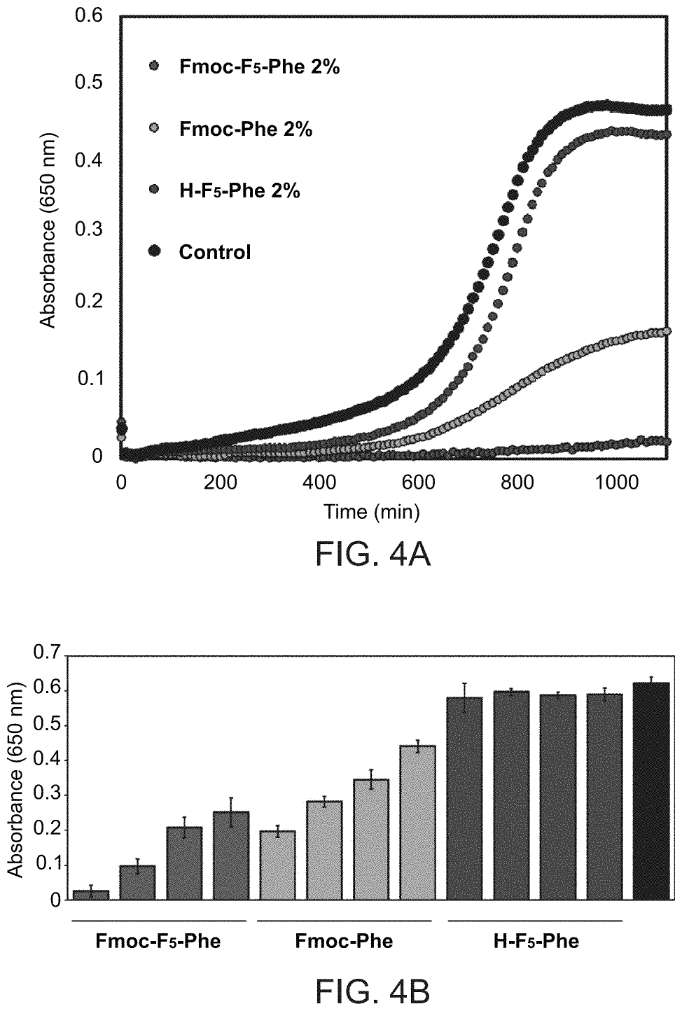

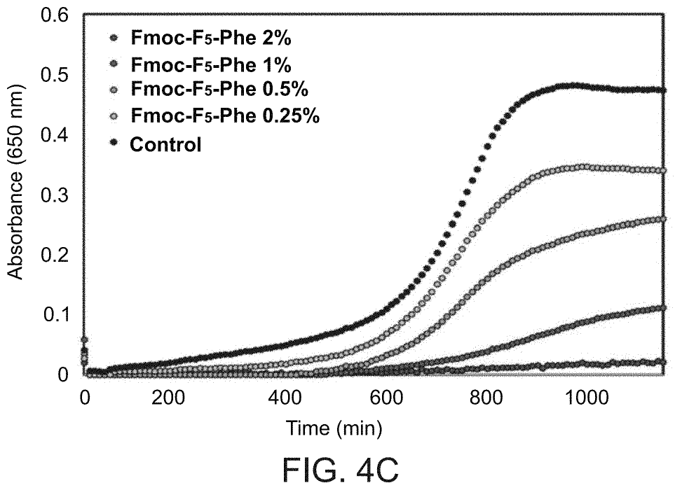

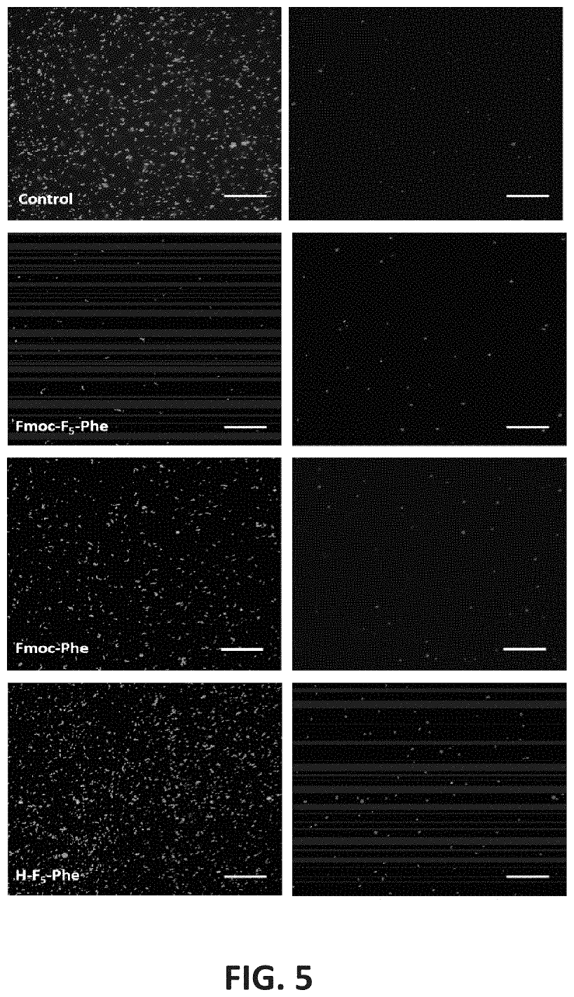

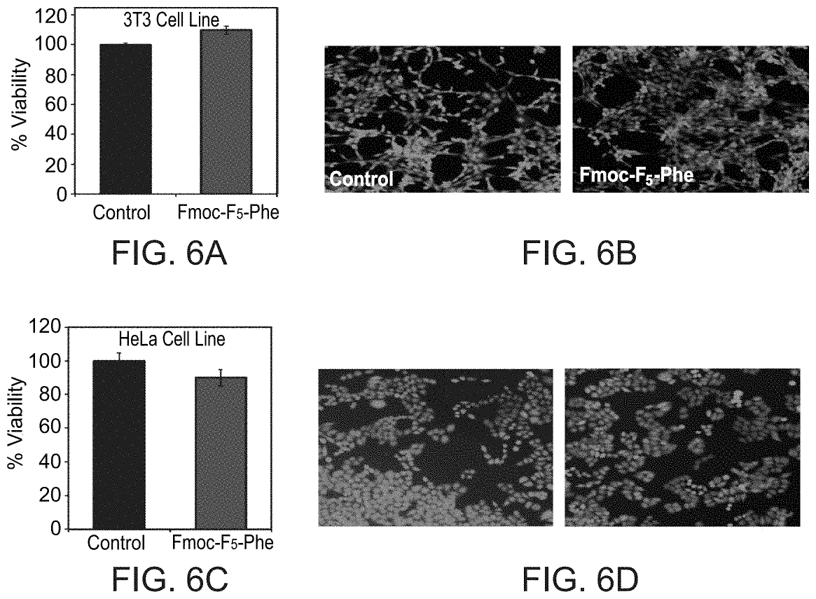

FIG. 1J presents the data obtained in bacterial membrane permeation evaluation following overnight growth using the SYTOX Blue-based membrane permeation assay. Blue fluorescence of the SYTOX Blue indicates bacterial cells with a compromised membrane. Upper panel presents data for control bacteria and lower panel for bacteria treated with 2 mM Fmoc-F5-Phe nanostructures.

FIGS. 2A-D demonstrate the incorporation and even distribution of self-assembled nanostructures of Fmoc-Pentafluoro-Phe in an exemplary dental composite restorative (Filtek™) by images obtained by optical microscopy for non-modified Filtek™ (FIG. 2A) and for Filtek™ embedding Fmoc-F5-Phe nanostructures (FIG. 2B), and by EDX analysis of the distribution of the carbon (red), silicon (pink), oxygen (green) and fluoride (yellow) atoms within the control dental resin restorative (FIG. 2C) and the dental resin composite restoratives (FIG. 2D).

FIGS. 3A-B present bar graphs showing the effect of the incorporation of nanostructures made of Fmoc-Pentafluoro-Phenylalanine in an exemplary dental resin composite restorative, on the Fmax, as determined by the Shear-Punch Test (FIG. 3A) and on the diametral tensile strength (DTS). In FIG. 3A, Fmax represents the maximum applied force required to physically punch through each sample.

FIGS. 3C-D present photographs showing restoration of occlusal fissures with a control formulation (left) and nanostructures-containing restorative formulation (right) (FIG. 3C) and of a spectral characterization of the color of the control (left) and nanostructures-containing restorative formulation (right) obtained utilizing a Spectroshade Micro-MHT dental spectrophotometer normalized to the Vita classical color guide.

FIGS. 4A-B present comparative plots showing the antibacterial effect of Filtek™ alone, and of Filtek™ having incorporated therein exemplary self-assembled nanostructures according to the present embodiments, as observed by direct-contact kinetic analysis (FIG. 4A) and a bar graph showing the end point dose dependency analysis (FIG. 4B) on S. mutans.

FIG. 4C presents comparative plots showing the bacterial growth inhibition kinetics evaluated by turbidity analysis via absorbance readings at 650 nm following direct contact of S. mutans bacteria with restorative composite containing Fmoc-F5-nanostructures for 1 hour.

FIG. 5 presents the antibacterial effect of a dental resin composite restorative incorporating nanostructures made of Fmoc-pentafluoro-phenylalanine, Pentafluoro-phenylalanine and Fmoc-phenylalanine as determined using the Live/Dead backlight bacterial viability kit. Green fluorescence of the Syto9 probe indicates bacterial cells with an intact membrane, while red fluorescence of Propidium Iodide (PI) indicates dead bacterial cells.

FIG. 6A-D presents the biocompatibility of the nano structure incorporated resin composite restoratives. The biocompatibility was evaluated utilizing an MTT cell viability analysis as well as mammalian cell viability analysis utilizing a fluorescent live-dead staining assay containing fluorescein diacetate (staining live cells) and Propidium Iodide (indicating dead cells) for 3T3 fibroblasts (FIG. 6A) and HeLa cells (FIG. 6B).

### DESCRIPTION OF SPECIFIC EMBODIMENTS OF THE INVENTION

The present invention, in some embodiments thereof, relates to dentistry and, more particularly, but not exclusively, to compositions featuring an anti-bacterial and/or anti-biofilm formation activity which are usable in dental applications such as formation of dental restorative composite materials, which feature anti-bacterial activity, and to composite materials prepared therefrom. The present invention, in some embodiments thereof, further relates to compositions featuring an anti-bacterial and/or anti-biofilm formation activity and to uses thereof in inhibiting, reducing and/or preventing a bacterial load and/or in inhibiting, reducing and/or retarding biofilm formation in and/or on a substrate.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Currently used dental compositions are typically made of polymerizable materials (polymeric precursors) that polymerize upon application to a desirable site in the oral cavity to thereby form the dental composite materials (e.g., dental composite restorative). Attempts have been made to incorporate anti-bacterial agents in such compositions, yet, such an incorporation typically requires high load of the anti-bacterial agents and was shown to result in adverse effect on the mechanical properties and performance of the resulting dental composite material.

The present inventors have devised and successfully practiced the incorporation of self-assembled nanostructures, made of aromatic amino acids and/or short peptides containing same, in dental compositions such as curable compositions that are usable for forming dental composite materials upon application. The present inventors have shown that the resulting composite material exhibits an anti-bacterial and anti-fouling (anti-biofilm formation; ABF) activity, while substantially retaining the mechanical properties (e.g., toughness, low thermal expansion), physical properties (e.g., a refractive index similar to that of natural teeth) and biocompatibility, that are attributed by the dental restorative material per se.

The present inventors have devised a methodology that enables efficient and homogenous incorporation of the self-assembled nanostructures in the curable composition. The nanostructures are fabricated separately prior to mixing with precursor curable composition, and are then mixed with the curable composition using certain techniques of agitation for achieving an effective dispersion.

While reducing the present invention to practice, the present inventors have successfully prepared self-assembled, typically fibrillar, nanostructures made of a plurality of aromatic molecules including, for example, free or N-protected, substituted or unsubstituted, phenylalanine, (see, FIGS. 1A-D). The present inventors have evaluated the antibacterial activity of exemplary such nanostructures, made of Fmoc-pentafluoro-phenylalanine (Fmoc-F5-Phe) (see, FIGS. 1E-J), and have developed incorporation methods for the self-assembled nanostructures within dental resin-based restorative compositions. See, for example, Example 2 in the Examples section that follows and FIGS. 2A-D.

The present inventors have demonstrated the potent antibacterial activity of the Fmoc-F5-Phe nanostructures. See, for example, FIGS. 1E-J.

The present inventors have demonstrated the potent antibacterial capabilities of restorative composite materials incorporating nanostructures formed of self-assembled Fmoc-F5-Phe units at an increasing loading dose of up to 2% by weight, which is substantially low in comparison to that of other antibacterial dental nano-assemblies, against S. mutans. See, FIGS. 4A-C and 5.

The present inventors have also demonstrated that the Fmoc-F5-Phe enhanced restorative composite materials are both biocompatible (see FIGS. 6A-D) and can be considered non-leachable materials (see Example 2), with the antibacterial effect stemming from the direct contact of bacterial cells with the restorative composite materials.

The potent antibacterial activity of the Fmoc-F5-Phe nanostructures and their simplified chemical synthesis, high availability and ease of incorporation into resin-based restorative compositions renders the resulting amalgamated antimicrobial dental resin restorative composite materials exceptionally suitable for clinical applications.

As exemplified in the Examples section that follows, a fluoride decorated self-assembling single amino acid-based building block, Fmoc-F5-Phe, was tested as an exemplary building block for forming self-assembled nanostructures. Following solvent-switch based nanostructure formation of Fmoc-F5-Phe, flexible, non-branched, fibrillary structures of 10 nm in width were observed via scanning electron microscopy. The nanostructures were then manually incorporated into a pre-polymerized (polymeric precursor curable formulation) Filtek™ Ultimate Flow dental resin composite restorative (3M-ESPE), a widely used dental restorative composition which does not display inherent antimicrobial capabilities, by manual mixing, sonication and centrifugation. The obtained amalgamated resin composition was subsequently polymerized (cured) by visible blue light. The incorporation of the nano-scale assemblies did not affect the coloring of the obtained amalgamated resin composite restoratives, an esthetically important feature. This incorporation process yielded a uniform and even distribution of the nanostructures within the amalgamated restorative, as demonstrated by energy-dispersive X-ray spectroscopy (EDX) analysis and optical microscopy (see, FIGS. 3A-D).

In order to evaluate the antimicrobial capabilities of the resin composite restoratives while simulating its clinical use, a direct-contact test (DCT) was carried out. This spectroscopic microplate reader based test, designed for compounds that are non-diffusible and non-soluble in water, allows measuring the effect of direct contact between the evaluated material and bacterial viability and growth. Four different W/W % samples of the resin composite material were evaluated at 0.25, 0.5, 1 and 2% Fmoc-F5-Phe nano-structure concentrations, and Filtek™ Ultimate Flow with no Fmoc-F5-Phe nano-structures additives, treated in the same manner, served as a control. Streptococcus mutans (S. mutans) was chosen for this evaluation as this strain is commonly found in the human oral cavity and is a significant caries-causing pathogen. Following direct contact of S. mutans bacteria with the Fmoc-F5-Phe incorporated materials the subsequent proliferation of the bacteria, was evaluated by optical density measurements over eighteen hours. The samples containing 0.25-1% nanostructures were able to inhibit bacterial growth in a dose dependent manner while 2% Fmoc-F5-Phe nano-structures were able to cause substantial (over 95%) bacterial growth inhibition and bacterial cell death, as evidenced by Live/Dead bacterial viability analysis.

Embodiments of the present invention provide antimicrobial dental compositions which are characterized by high purity, low cost and efficient and scalable method of preparation.

Embodiments of the present invention further provide antimicrobial dental composites prepared from the antimicrobial compositions which are characterized by high purity, low cost and efficient and scalable method of preparation.

Embodiments of the present invention further provide antimicrobial compositions which can be efficiently incorporated in polymeric matrices usable in manufacturing articles such as medical devices and food packages, and which can benefit from the antibacterial capabilities of the compositions.

Herein throughout, the expressions “dental composite restorative”, “dental restorative composite material”, “dental restorative material” and grammatical diversions thereof, all refer to the final material used as dental restorative, typically upon application of resin-based material and hardening thereof. These materials are also referred to herein and in the as art as “dental sealant”.

Herein throughout, the terms “curable composition” or “curable dental composition” or “dental restorative composition”, “curable formulation”, “curable dental formulation” and “dental restorative formulation” are used interchangeably and describe the precursor composition that is applied to an oral cavity, and which forms, when hardened (e.g., cured, polymerized), a dental composite, or a dental composite material as described herein.

Dental Composition:

According to an aspect of some embodiments of the present invention there is provided a composition comprising a dental formulation and at least one self-assembled nanostructure associated with the dental formulation. This composition is also referred to herein as a dental composition, and in some embodiments can be a dental restorative composition, etc., as described herein.

According to some of any of the embodiments described herein, the composition comprises a plurality of self-assembled nanostructures, that is, two or more, and preferably dozens or more, self-assembled nanostructures, which can be the same or different and which comprise at least one self-assembled nanostructure that is formed of a plurality of aromatic moieties, as described herein. According to some of any of the embodiments described herein, the composition comprises a plurality of self-assembled nanostructures and at least a portion of these self-assembled nanostructures, are nanostructures made of a plurality of aromatic moieties, as described herein, which can be the same or different.

According to some of any of the embodiments described herein, the composition comprises a plurality of self-assembled nanostructures and all of the self-assembled nanostructures are nanostructures made of a plurality of aromatic moieties, as described herein, which can be the same or different.

By “at least a portion” it is meant at least 5%, or at least 10%, or at least 20%, or at least 30%, or at least 40%, preferably at least 50 5, at least 60%, at least 70%, at least 80%, at least 90%, at least 95%, at least 98%, and up to 100% (all) of the plurality of nanostructures.

By “associated with” it is meant that the one or more nanostructures are incorporated in and/or on the dental formulation, as described herein, and interact with the formulation by physical (as being dispersed, embedded, incorporated, entangled, etc., in and/or on the formulation) and/or chemical interactions (e.g., covalent, electrostatic, hydrogen bond, Van der Waals and/or aromatic interactions).

According to some of any of the embodiments described herein, the composition comprises a plurality of nanostructures that are dispersed in the dental formulation.

According to some of any of the embodiments described herein, the nanostructures are dispersed evenly and homogeneously in the dental formulation.

According to some of any of the embodiments described herein, the dental formulation is a curable dental formulation, as described herein, and the nanostructures are dispersed evenly and homogeneously in the dental formulation, as measured and shown, for example, in FIGS. 2A-D.

Self-assembled nanostructures: According to the present embodiments, the nanostructures are self-assembled nanostructures, and according to some of these embodiments, the nanostructures are self-assembled upon forming aromatic interactions between the aromatic portion of the aromatic molecules that form the nanostructures.

According to some of any of the embodiments described herein the composition comprises a plurality of nanostructures, and in at least a portion of the plurality of nanostructures, each nanostructure is formed of a plurality of aromatic molecules.

Each nanostructure in the plurality of nanostructures can independently include one or more types of aromatic molecules.

In some embodiments, one portion of the plurality of nanostructures can be made of one type of aromatic molecules, and another portion of the plurality of nanostructures can be made of another type of aromatic molecules, and so forth, such that when two or more nanostructures are included in the composition, the nanostructures can be the same or different.

In some embodiments, all of the nanostructures are made of the same one or more types of aromatic molecules.

According to some of any of the embodiments described herein, each of the aromatic molecules comprises an aromatic amino acid.

By “aromatic molecule” it is meant a molecule (a compound) that comprises at least one aromatic moiety or group.

As used herein, the phrase “aromatic group” or “aromatic moiety” describes a monocyclic or polycyclic moiety having a completely conjugated pi-electron system. The aromatic group can be an all-carbon moiety or can include one or more heteroatoms such as, for example, nitrogen, sulfur or oxygen. The aromatic group can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine.

Exemplary aromatic groups include, for example, phenyl, biphenyl, naphthalenyl, phenanthrenyl, anthracenyl, [1,10]phenanthrolinyl, indoles, thiophenes, thiazoles and, [2,2′]bipyridinyl, each being optionally substituted. Thus, representative examples of aromatic groups that can serve as the side chain within the aromatic amino acid described herein include, without limitation, substituted or unsubstituted naphthalenyl, substituted or unsubstituted phenanthrenyl, substituted or unsubstituted anthracenyl, substituted or unsubstituted [1,10ϕphenanthrolinyl, substituted or unsubstituted [2,2′]bipyridinyl, substituted or unsubstituted biphenyl and substituted or unsubstituted phenyl. The aromatic group can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

In some of any of the embodiments described herein, the aromatic molecule comprises at least one aromatic moiety that is an all-carbon aromatic moiety, e.g., an aryl as defined herein.

In some of any of the embodiments described herein, the aromatic molecule is or comprises an aromatic amino acid.

In some of any of the embodiments described herein, the aromatic molecule is an aromatic amino acid.

By “aromatic amino acid” it is meant an amino acid, or an amino acid residue in a peptide comprising same, that has an aromatic moiety or group, as defined herein, is its side chain. In exemplary embodiments, an aromatic amino acid has, for example, a substituted or unsubstituted naphthalenyl or a substituted or unsubstituted phenyl, in its side chain. The substituted phenyl may be, for example, pentafluoro phenyl, iodophenyl, biphenyl and nitrophenyl.

According to some of any of the embodiments described herein, in at least one nanostructure, or in at least a portion of a plurality of nanostructures, or in each nanostructure in a plurality of nanostructures, in at least a portion, or in all, of the plurality of aromatic molecules forming the nanostructure, each of the aromatic molecules is or comprises an aromatic amino acid.

According to some of any of the embodiments described herein, in at least one nanostructure, or in at least a portion of a plurality of nanostructures, or in each nanostructure in a plurality of nanostructures, in at least a portion, or in all, of the plurality of aromatic molecules forming the nanostructure, each of the aromatic molecules comprises an aromatic amino acid having an end-capping moiety attached thereto.

The phrase “end-capping moiety”, as used herein, refers to a moiety that when attached to the terminus of a peptide, modifies the end-capping. The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996).

In the context of the present embodiments, an end-capping moiety can be attached to alpha-amine or alpha-carboxylic group of an amino acid, thus forming an end-capped amino acid, or end-capping modified amino acid.

End-capping moieties that are described in the context of peptides as suitable for capping the N-terminus of a peptide are suitable in the context of some of the present embodiments as moieties that are attached to an alpha amine of an end-capped amino acid (e.g., an aromatic amino acid).

End-capping moieties that are described in the context of peptides as suitable for capping the C-terminus of a peptide are suitable in the context of the present embodiments as moieties that are attached to an alpha carboxylic acid of an end-capped amino acid (e.g., an aromatic amino acid).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denote d herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”).

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively, the —COOH group of the C-terminus end-capping may be modified to an amide group.

Other end-capping modifications include replacement of the amine and/or carboxyl with a different moiety, such as hydroxyl, thiol, halide, alkyl, aryl, alkoxy, aryloxy and the like, as these terms are defined hereinbelow.

In some embodiments of the present invention, a nanostructure is made of a plurality of aromatic amino acids and all of the aromatic amino acids composing the nanostructure are end-capping modified. In some of these embodiments, the aromatic amino acids are modified only at the alpha-amine or the alpha-carboxylic acid thereof, resulting in a nanostructure that has a negative net charge or a positive net charge, respectively. In another embodiment, the aromatic amino acids are modified at both the alpha amine and the alpha carboxylic acid, resulting in an uncharged nanostructure.

According to some of any of the embodiments described herein, an aromatic amino acid is end-capping modified at the alpha-amine thereof.

According to some of any of the embodiments described herein, when an aromatic amino acid is end-capping modified, the end capping moiety is an aromatic end-capping moiety.

According to some of any of the embodiments described herein, an aromatic amino acid is end-capping modified at the alpha-amine thereof, and the end-capping moiety is an aromatic moiety or a non-aromatic moiety.

Representative examples of aromatic end capping moieties suitable for N-terminus modification, or alpha-amine modification, include, without limitation, fluorenylmethyloxycarbonyl (Fmoc). Representative examples of non-aromatic end capping moieties suitable for C-terminus modification include, without limitation, benzyl, benzyloxycarbonyl (Cbz), trityl and substituted trityl groups.

Representative examples of non-aromatic end capping moieties suitable for N-terminus modification or alpha-amine modification, include, without limitation, formyl, acetyl trifluoroacetyl, tert-butoxycarbonyl, trimethylsilyl, and 2-trimethylsilyl-ethanesulfonyl. Representative examples of non-aromatic end capping moieties suitable for C-terminus modification include, without limitation, amides, allyloxycarbonyl, trialkylsilyl ethers and allyl ethers.

According to some of any of the embodiments described herein, an aromatic amino acid is end-capping modified at the alpha-amine thereof, and the end-capping moiety is an aromatic moiety.

In some of any of the embodiments described herein, the end-capping moiety is an aromatic end-capping moiety.

In some of any of the embodiments described herein, the end-capping moiety is attached to the alpha-amine of the aromatic amino acid.

According to some of any of the embodiments described herein, in at least a portion, or in all, of the plurality of aromatic molecules, each of the aromatic molecules comprises a peptide of from 2 to 6 amino acid residues, and at least one of the amino acid residues is an aromatic amino acid as described herein in any of the respective embodiments.

In some of these embodiments, the peptide is a dipeptide, and in some embodiments it is a homo-dipeptide.

In some of these embodiments, the peptide is an end-capping modified peptide.

According to some embodiments, the end-capping modified peptides are dipeptides, i.e., having two amino acid residues, and according to some embodiments, the end-capping modified dipeptides is a homodipeptides, having two amino acid residues which are identical with respect to their side-chains residue.

Representative examples of such end-capping modified homodipeptides include, without limitation, an end-capping modified naphthylalanine-naphthylalanine (Nal-Nal) dipeptides, end-capping modified (pentafluro-phenylalanine)-(pentafluro-phenylalanine) dipeptides, end-capping modified (iodo-phenylalanine)-(iodo-phenylalanine), end-capping modified (4-phenyl phenylalanine)-(4-phenyl phenylalanine) and end-capping modified (p-nitro-phenylalanine)-(p-nitro-phenylalanine).

Thus, also contemplated are homodipeptides, and more preferably aromatic homodipeptides in which each of the amino acids comprises an aromatic moiety, such as, but not limited to, substituted or unsubstituted naphthalenyl and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine

When substituted, the phenyl, naphthalenyl or any other aromatic moiety includes one or more substituents such as, but not limited to, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

In some of any of these embodiments, the end-capping modified peptide is an N-terminus modified peptide.

In some of any of these embodiments, the end-capping modified peptide comprises an aromatic end-capping moiety, as described herein.

In some of any of the embodiments described herein, the aromatic end-capping moiety is

Fmoc.

In some of any of the embodiments described herein, at least one, or at least a portion, or each nanostructure in the composition is formed, and is made, of a plurality of end-capping modified aromatic amino acids as described herein in any of the respective embodiments.

In some of any of the embodiments described herein, at least one, or at least a portion, or each nanostructure in the composition is formed, and is made, of a plurality of end-capping modified aromatic dipeptides (in which at least one, preferably both, of the amino acid residues is an aromatic amino acid residue).

In some of any of the embodiments described herein, at least one, or at least a portion, or each nanostructure in the composition is formed, and is made, of a plurality of aromatic amino acids.

In some of any of the embodiments described herein, the aromatic amino acid is phenylalanine.

In some of any of the embodiments described herein, at least one, or at least a portion, or each nanostructure in the composition is formed, and is made, a plurality of phenylalanine molecules.

In some of any of the embodiments described herein, in at least a portion, or in all, of the aromatic molecules, the aromatic amino acid is a halogenated aromatic amino acid, comprising a halogenated aromatic moiety in its side chain.

In some of any of the embodiments described herein, at least one, or at least a portion, or each nanostructure in the composition is formed, and is made, of a plurality of halogenated aromatic amino acid molecules.

In embodiments where the aromatic molecule is a peptide, at least one amino acid residue in the peptide is a halogenated aromatic amino acid, comprising a halogenated aromatic moiety in each side chain.

In some of any of the embodiments described herein, at least one, or at least a portion, or each nanostructure in the composition is formed, and is made, of a plurality of peptides, as described herein in any of the respective embodiments, in which at least one amino acid residue is halogenated aromatic amino acid as described herein.

In some of any of the embodiments described herein, the halogenated aromatic amino acid comprises in its side chain an aromatic moiety substituted by 1, 2, 3, 4, 5 or more halogen substituents.

In some of any of the embodiments described herein, the halogenated aromatic amino acid is a fluorinated aromatic amino acid.

In some of any of the embodiments described herein, the halogenated aromatic amino acid is a halogenated phenylalanine.

In some of any of the embodiments described herein, at least one, or at least a portion, or each nanostructure in the composition is formed, and is made, of a plurality of halogenated phenylalanine molecules.

In some of any of the embodiments described herein, at least one, or at least a portion, or each nanostructure in the composition is formed, and is made, of a plurality of halogenated aromatic amino acid molecules.

In some of any of the embodiments described herein, at least one, or at least a portion, or each nanostructure in the composition is essentially consisted of a plurality of halogenated aromatic amino acid molecules, e. g., halogenated phenylalanine molecules.

Nanostructures made of a plurality of halogenated aromatic acid acid molecules, according to any one of the respective embodiments, are collectively referred to herein as such.

In some of any of the embodiments described herein for nanostructures made of a plurality of halogenated aromatic acid acid molecules, at least a portion, and preferably each, of the halogenated aromatic acid molecules in an end-capping modified molecule, and in some of these embodiments, the molecule is modified at the alpha-amine thereof, preferably, but not obligatory, by an aromatic end-capping moiety as described herein.

In some of any of the embodiments described herein for nanostructures made of a plurality of halogenated aromatic acid acid molecules, at least a portion, and preferably each, of the halogenated aromatic acid molecules is a modified halogenated aromatic amino acid having an aromatic end capping moiety as described herein, for example, Fmoc, attached to its alpha amine.

In some of any of the embodiments described herein, a halogenated phenylalanine, or a halogenated aromatic amino acid, comprises 1, 2, 3, 4 or 5 substituents on the aromatic moiety, and at least one of these substituents is halo. When two of or more of substituents are halo, the halo can be the same of different. In some of these embodiments, at least one of the halo substituents is fluoro.

In some of any of the embodiments described herein, the halogenated aromatic amino acid is pentafluoro-phenylalanine.

In some of any of the embodiments described herein, at least one, or at least a portion, or each nanostructure in the composition is formed, and is made, of a plurality of pentafluoro-phenylalanine molecules.

In some of any of the embodiments described herein, at least one, or at least a portion, or each nanostructure in the composition, consists essentially of pentafluoro-phenylalanine molecules.

In some of any of the embodiments described herein, at least one, or at least a portion, or each nanostructure in the composition, consists essentially of Fmoc-pentafluoro-phenylalanine molecules.

In some of any of the embodiments described herein, the halogenated aromatic amino acid is an end-capping modified amino acid, and in some embodiments, it is modified by an aromatic end-capping moiety. In some of these embodiments, the alpha-amine of the halogenated aromatic amino acid is modified by an aromatic end-capping moiety.

In some of any of the embodiments described herein, the plurality of aromatic molecules comprises a plurality of Fmoc-pentafluoro-phenylalanine.

In some of any of the embodiments described herein, at least one, or at least a portion, or each nanostructure in the composition is formed, and is made, of a plurality of phenylalanine molecules.

In some of any of the embodiments described herein, the halogenated aromatic amino acid is an end-capping modified amino acid, and in some embodiments, it is modified by an aromatic end-capping moiety. In some of these embodiments, the alpha-amine of the aromatic amino acid is modified by an aromatic end-capping moiety.

In some of any of the embodiments described herein, the plurality of aromatic molecules comprises a plurality of Fmoc-phenylalanine.

The phrase “aromatic dipeptide” describes a peptide composed of two amino acid residues, at least one, and preferably both, being an aromatic amino acid as defined herein.

In some embodiments, the aromatic dipeptide comprises an aromatic group which is unsubstituted or which is substituted by one or more substituents other than halogen.

The phrase “end-capping modified dipeptide”, as used herein, refers to a dipeptide as described herein which has been modified at the N-(amine)terminus and/or at the C-(carboxyl)terminus thereof. The end-capping modification refers to the attachment of a chemical moiety to the terminus, so as to form a cap. Such a chemical moiety is referred to herein as an end-capping moiety and is typically also referred to herein and in the art, interchangeably, as a peptide protecting moiety or group.

In a preferred embodiment of the present invention, the end-capping modified dipeptides are modified by an aromatic (e.g. Fmoc) end-capping moiety.

The end-capping moieties described herein for N-terminus modification can also be utilized for providing an amine-modified aromatic amino acid as described herein.

According to some of any of the embodiments described herein the at least one nanostructure is a fibrillary nanostructure.

As used herein the phrase “fibrillar nanostructure” refers to a filament or fiber having a diameter or a cross-section of less than 1 μm (preferably less than about 100 nm, more preferably less than about 50 nm, and even more preferably less than about 20 nm, e.g., of about 10 nm). The length of the fibrillar nanostructure is preferably at least 1 nm, more preferably at least 10 nm, even more preferably at least 100 nm and even more preferably at least 500 nm. In some embodiments, the fibrillar nanostructure described herein is characterized as non-hollowed or at least as having a very fine hollow.

In some of any of the embodiments described herein, the nanostructure exhibits an anti-microbial activity, and in some embodiments, it exhibits an anti-bacterial activity.

By “anti-microbial activity” it is meant that the nanostructure is capable of inhibiting, arresting or reducing the growth or the rate of growth of a microorganism, preferably a pathogenic microorganism and/or is capable of reducing a load of the microorganism is a substrate (which can be animate or non-animate substrate).

When the microorganism is a bacterium, the anti-microbial activity is anti-bacterial activity.

In some of any of the embodiments described herein, the nanostructure exhibits an anti-biofilm formation (ABF) activity, or anti-biofouling activity, and as such is capable of inhibiting, reducing or retarding a formation of a biofilm on a surface of a substrate (which can be animate or non-animate substrate).

The term “biofilm”, as used herein, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaea, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and fungi. The cells of a microorganism growing in a biofilm are physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

The phrases “anti-biofilm formation (ABF) activity” refers to the capacity of a substance to effect the prevention of formation of a biofilm of bacterial, fungal and/or other cells; and/or to effect a reduction in the rate of buildup of a biofilm of bacterial, fungal and/or other cells, on a surface of a substrate.

In some embodiments, the biofilm is formed of bacterial cells (or from a bacterium).

In some embodiments, a biofilm is formed of bacterial cells of bacteria selected from the group consisting of all Gram-positive and Gram-negative bacteria.

As used herein, the term “preventing” in the context of the formation of a biofilm, indicates that the formation of a biofilm is essentially nullified or is reduced by at least 20%, at least 30%, at least 40%, at least 50%, at least 60%, at least 70%, at least 80%, at least 90%, including any value therebetween, of the appearance of the biofilm in a comparable situation lacking the presence of nanostructure of the present embodiments. Alternatively, preventing means a reduction to at least 15%, 10% or 5% of the appearance of the biofilm in a comparable situation lacking the presence of the nanostructure. Methods for determining a level of appearance of a biofilm are known in the art.

In some embodiments, inhibiting, reducing and/or retarding a formation of a biofilm as described herein is reflected by reducing biofilm formation on the substrate's surface by at least 20%, at least 30%, at least 40%, at least 50%, at least 60%, at least 70%, at least 80%, at least 90%, including any value therebetween, compared to the same substrate when not treated with the nanostructure.

In some of any of the embodiments described herein, the nanostructure exhibits an anti-microbial, anti-bacterial and/or anti-biofouling activity when the substrate is a bodily organ or tissue, and in some of these embodiments, the substrate is an organ or tissue in the oral cavity.

Assays and methods for determining an anti-microbial, anti-bacterial and anti-biofouling activity are known and widely used in the art and all are contemplated herein for determining such an activity of a nanostructure.

Exemplary assays and methods are described in the Examples section that follows.

Anti-biofouling activity can be determined by methods that determine the capability of a substance to disrupt and/or penetrate bacterial membranes.

Dental formulation: According to some of any of the embodiments described herein, the dental formulation is a formulation that is intended for use in a dental application, by being contacted with an organ or tissue in the oral cavity. Any commercially available dental formulation is usable in the context of these embodiments.

According to some embodiments, the dental formulation is a mouth wash formulation, a tooth paste, a cream, a lotion, an ointment, or any other formulation that is configured to be applied to oral cavity.

According to some of any of the embodiments described herein, the dental formulation is a curable dental formulation.

Herein throughout, a “curable formulation” or a “curable material” is a formulation or a material or a mixture of materials which, when exposed to a curing condition (e.g., curing energy), as described herein, solidifies or hardens to form a cured material as defined herein. Curable materials are typically polymerizable materials, which undergo polymerization and/or cross-linking when exposed to a suitable energy source or any other curing condition. A curable material or formulation is typically such that its viscosity increases by at least one order of magnitude when it is exposed to a curing condition.

As used herein, the term “curing” or “hardening” describes a process in which a formulation is hardened. This term encompasses polymerization of monomer(s) and/or oligomer(s) and/or cross-linking of polymeric chains (either of a polymer present before curing or of a polymeric material formed in a polymerization of the monomers or oligomers). The product of a curing reaction or of a hardening is therefore typically a polymeric material and in some cases a cross-linked polymeric material.

Herein, the phrase “a condition that affects curing” or “a condition for inducing curing”, which is also referred to herein interchangeably as “curing condition” or “curing inducing condition” describes a condition which, when applied to a formulation that contains a curable material, induces polymerization of monomer(s) and/or oligomer(s) and/or cross-linking of polymeric chains. Such a condition can include, for example, application of a curing energy, as described hereinafter, to the curable material(s), and/or contacting the curable material(s) with chemically reactive components.

A “curing energy” typically includes application of radiation or application of heat. The radiation can be electromagnetic radiation (e.g., ultraviolet or visible light), or electron beam radiation, or ultrasound radiation or microwave radiation, depending on the materials to be cured. The application of radiation (or irradiation) is effected by a suitable radiation source. For example, an ultraviolet or visible or infrared or Xenon lamp can be employed.

A curable material or system that undergoes curing upon exposure to radiation is referred to herein interchangeably as “photopolymerizable” or “photoactivatable” or “photocurable”.

In some of any of the embodiments described herein, a curable material is a photopolymerizable material, which polymerizes or undergoes cross-linking upon exposure to radiation, as described herein, and in some embodiments the curable material is a UV-curable material, which polymerizes or undergoes cross-linking upon exposure to UV-vis radiation, as described herein.

In some embodiments, a curable material as described herein includes a polymerizable material that polymerizes via photo-induced radical polymerization.

When the curing energy comprises heat, the curing is also referred to herein and in the art as “thermal curing” and comprises application of thermal energy.

A curable material or system that undergoes curing upon exposure to heat is referred to herein as “thermally-curable” or “thermally-activatable” or “thermally-polymerizable”.

A curing condition can also be contacting a curable material or formulation with an environment that exhibits conditions that affect curing. For example, a pH that affects a pH-sensitive polymerization, or a presence of a chemical agent that promotes polymerization and/or cross-linking (e.g., an agent present in the saliva).

According to some of any of the embodiments described herein, the curable dental formulation is configured for forming a polymeric matrix for dental application, that is, is such that forms a polymeric matrix usable in dental applications, when applied to an area in the oral cavity (e.g., tooth, root canal, gum, etc.), typically upon hardening (e.g., by being subjected to a suitable curing condition).

According to some of any of the embodiments described herein, the curable dental formulation comprises a polymeric precursor, and is also referred to herein as a polymeric precursor formulation or mixture, and in some of these embodiments it is configured for forming a dental polymeric composite, or a dental composite material such as a dental restorative material, or is such that forms a dental polymeric or composite material when applied to an area in the oral cavity, typically upon being subjected to a suitable curing condition.

In some embodiments, the polymeric precursor formulation can include polymerizable materials, optionally along with polymerization initiators, or can include polymeric materials which harden upon application in the oral cavity.

In some embodiments, the term “precursor” as used herein encompasses any material or mixture that forms a polymeric matrix for dental application when applied to an area in the oral cavity (e.g., tooth, gums, etc.), optionally in combination with an agent that promotes polymerization (e.g., an initiator or photoinitiator).

According to some of any of the embodiments described herein, the polymeric precursor formulation comprises at least one precursor (polymerizable) molecule selected from a precursor of a polyacrylate, a precursor of a polymethacrylate and a precursor of a polyurethane, optionally in combination with an agent that promotes polymerization (e.g., an initiator or photoinitiator). Epoxy polymeric precursors are also contemplated.

According to some of any of the embodiments described herein, the polymeric precursor mixture comprises at least two or all of the above-mentioned precursor molecules. Any of the known polymeric precursor formulations that are usable in dental applications, including commercially available products, are usable in the context of the t embodiments related to curable dental formulations.

According to some embodiments, the polymeric precursor formulation encompasses any commercially available or costumely-prepared formulation that is usable for providing dental adhesives, bone cement, dental restorative materials such as all types of composite based materials for filling tooth-decay cavities, endodontic filling materials (cements and fillers) for filling the root canal space in root canal treatment, for providing materials used for provisional and final tooth restorations or tooth replacement, including but not restricted to inlays, onlays, crowns, partial dentures (fixed or removable) dental implants, and permanent and temporary cements used in dentistry for various known purposes, dental resin based cements, dental sealers, dental composite materials, dental adhesives and cements, dental restorative composites, bone cements, and tooth pastes. Also contemplated are formulations usable for forming a varnish or glaze which is applied to the tooth surface, a restoration of tooth or a crown. In some of any of the embodiments described herein, the composition is formulated for administration/application to an oral cavity, e.g., to a tooth, root canal, a gum.

The composition may be formulated as a tooth paste, and/or may be applied as a denture cleaner, a post hygienic treatment dressing or gel, a mucosal adhesive paste, a dental adhesive, a dental restorative composite based material for filling tooth, decay cavities, a dental restorative endodontic filling material for filling root canal space in root canal treatment, a dental restorative material used for provisional and final tooth restorations or tooth replacement, a dental inlay, a dental onlay, a crown, a partial denture, a complete denture, a dental implant and a dental implant abutment.

In exemplary embodiments, the polymeric precursor formulation is or comprises any dental curable formulation or composition that is known as usable as a dental composite resin, a dental adhesive, a dental resin cement, a dental glass ionomer cement, a dental resin-modified glass ionomer cement, and a dental quick cure resin, which are used in a dental restorative filling material, a dental adhesive material, a dental luting material, a dental temporary sealing material, a dental provisional crown material, and/or a dental pit and fissure sealant.

In some of any of the embodiments described herein, the polymeric precursor formulation comprises one or more curable (e.g., polymerizable and/or cross-linkable) material(s) and a polymerization initiator and/or a polymerization accelerator.

In some of any of the embodiments described herein the polymerizable material is such that has one or more functional polymerizable unsaturated group such as a (meth)acryloyl group, a vinyl group, or a styrene group. Exemplary materials include, but are not limited to, (meth)acrylic acid ester or a (meth)acrylamide derivative. The expression “(meth)acryl” is used to include both methacryl and acryl. Exemplary mono-functional (meth)acrylic acid esters or (meth)acrylamide derivatives include methyl (meth)acrylate, isobutyl (meth)acrylate, benzyl (meth)acrylate, lauryl (meth)acrylate, 2-(N,N-dimethylamino)ethyl (meth)acrylate, 2,3-dibromopropyl (meth)acrylate, 2-hydroxyethyl (meth)acrylate, 6-hydroxyhexyl (meth)acrylate, 10-hydroxydecyl (meth)acrylate, propylene glycol mono(meth)acrylate, glycerin mono(meth)acrylate, erythritol mono(meth)acrylate, N-methylol (meth)acrylamide, N-hydroxyethyl (meth)acrylamide, N-(dihydroxyethyl) (meth)acrylamide, (meth)acryloyloxydodecylpyridinium bromide, (meth)acryloyloxydodecylpyridinium chloride, (meth)acryloyloxyhexadecylpyridinium chloride, (meth)acryloyloxydecylammonium chloride, and 10-mercaptodecyl (meth)acrylate.

Exemplary aromatic-based di-functional polymerizable materials include: 2,2-bis((meth)acryloyloxyphenyl)propane, 2,2-bis [4-(3-(meth)acryloyloxy-2-hydroxypropoxy) phenyl] propane (generally called “Bis-GMA”), 2,2-bis(4-(meth)acryloyloxyethoxyphenyl) propane, 2,2-bis(4-(meth)acryloyloxypolyethoxyphenyl)propane, 2,2-bis(4-(meth) acryloyloxydiethoxyphenyl)propane, 2,2-bis(4-(meth)acryloyloxytriethoxyphenyl)propane, 2,2-bis(4-(meth)acrylo yloxytetraethoxyphenyl)propane, 2,2-bis(4-(meth) acryloyloxypentaethoxyphenyl)propane, 2,2-bis(4-(meth)acryloyloxydipropoxyphenyl)propane, 2-(4-(meth)acryloyloxydiethoxyphenyl)-2-(4-(meth)acryloyloxyethoxyphenyl)-propane, 2-(4-(meth)acryloyloxydiethoxyphenyl)-2-(4-(meth)acryloyloxytriet-hoxyphenyl)propane, 2-(4-(meth)acryloyloxydipropoxyphenyl)-2-(4-(meth)acryloyloxytriethoxyphenyl)propane, 2,2-bis(4-(meth)acryloyloxypropoxyphenyl)propane, 2,2-bis(4-(meth)acryloyloxyisopropoxyphenyl) propane, and 1,4-bis(2-(meth)acryloyloxyethyl) pyromellitate.

Exemplary aliphatic-based difunctional polymerizable materials include erythritol di(meth)acrylate, sorbitol di(meth)acrylate, mannitol di(meth)acrylate, pentaerythritol di(meth)acrylate, dipentaerythritol di(meth)acrylate, glycerol di(meth)acrylate, ethylene glycol di(meth)acrylate, diethylene glycol di(meth)acrylate, triethylene glycol di(meth)acrylate, propylene glycol di(meth)acrylate, butylene glycol di(meth)acrylate, neopentyl glycol di(meth)acrylate, polyethylene glycol di(meth)acrylate (particularly polyethylene glycol di(meth)acrylate having nine or more oxyethylene groups), 1,3-butanediol di(meth)acrylate, 1,5-pentanediol di(meth)acrylate, 1,6-hexane diol di(meth)acrylate, 1,10-decanediol di(meth)acrylate, 2,2,4-trimethylhexamethylene bis(2-carbamoyloxyethyl) dimethacrylate (generally called “UDMA”), and 1,2-bis(3-methacryloyloxy-2-hydroxypropyloxy)ethane.

Exemplary tri- or higher-functional polymerizable materials include trimethylolpropane tri(meth)acrylate, trimethylolethane tri(meth)acrylate, trimethylolmethane tri(meth)acrylate, pentaerythritol tri(meth)acrylate, pentaerythritol tetra(meth)acrylate, dipentaerythritol tri(meth)acrylate, dipentaerythritol tetra(meth)acrylate, dipentaerythritol penta(meth)acrylate, N,N-(2,2,4-trimethylhexamethylene)bis [2-(aminocarboxy)propane-1,3-diol[te-tramethacrylate, and 1,7-diacryloyloxy-2,2,6,6-tetraacryloyloxymethyl-4-oxyheptane.

Exemplary polymerization initiators can be selected from those used in general industry can be used as the polymerization initiators, preferably those known for dental use.

Exemplary initiators include a combination of an oxidant and a reductant used as a chemical polymerization initiator.

Examples of the oxidant include organic peroxides, azo compounds, and inorganic peroxides.

Examples of the organic peroxides include diacyl peroxides, peroxyesters, peroxycarbonates, dialkyl peroxides, peroxyketals, ketone peroxides, and hydroperoxides. Specific examples of the diacyl peroxides include benzoyl peroxide, 2,4-dichlorobenzoyl peroxide, m-toluoyl peroxide, and lauroyl peroxide. Specific examples of the peroxyesters include t-butyl peroxybenzoate, bis-t-butyl peroxyisophthalate, and t-butyl peroxy-2-ethylhexanoate. Specific examples of the peroxycarbonates include t-butyl peroxy isopropyl carbonate. Specific examples of the dialkyl peroxides include dicumyl peroxide, di-t-butyl peroxide, and 2,5-dimethyl-2,5-bis(benzoylperoxy)hexane. Specific examples of the peroxyketals include 1,1-bis(t-butylperoxy)-3,3,5-trimethylcyclohexane, 1,1-bis(t-butylperoxy)cyclohexane, and 1,1-bis(t-hexylperoxy)cyclohexane. Specific examples of the ketone peroxides include methyl ethyl ketone peroxide, cyclohexanone peroxide, and methyl acetoacetate peroxide. Specific examples of the hydroperoxides include t-butyl hydroperoxide, cumene hydroperoxide, diisopropylbenzene hydroperoxide, and 1,1,3,3-tetramethylbutyl hydroperoxide.

Examples of the azo compounds include azobisisobutyronitrile and azobisisobutylvaleronitrile.

Examples of the inorganic peroxides include sodium persulfate, potassium persulfate, aluminum persulfate, and ammonium persulfate.

Examples of the reductant include aromatic amines having no electron-withdrawing group in the aromatic ring, thioureas, and ascorbic acid.

Examples of the aromatic amines include N,N-bis(2-hydroxyethyl)-3,5-dimethylaniline, N,N-bis(2-hydroxyethyl)-p-toluidine, N,N-bis(2-hydroxyethyl)-3,4-dimethylaniline, N,N-bis (2-hydroxyethyl)-4-ethylaniline, N,N-bis (2-hydroxyethyl)-4-isopropylaniline, N, N-bis(2-hydroxyethyl)-4-t-butylaniline, N,N-bis(2-hydroxyethy)-3,5-di-isopropylaniline, N,N-bis(2-hydroxyethyl)-3,5-di-t-butylaniline, N, N-dimethylaniline, N,N-dimethyl-p-toluidine, N,N-dimethyl-m-toluidine, N,N-diethyl-p-toluidine, N,N-dimethyl-3,5-dimethylaniline, N,N-dimethyl-3,4-dimethylaniline, N, N-dimethyl-4-ethylaniline, N, N-dimethyl-4-isopropylaniline, N, N-dimethyl-4-t-butylaniline, and N,N-dimethyl-3,5-di-t-butylaniline.

Examples of the thioureas include thiourea, methylthiourea, ethylthiourea, N,N′-dimethylthiourea, N,N′-diethylthiourea, N,N′-di-n-propylthiourea, dicyclohexylthiourea, trimethylthiourea, triethylthiourea, tri-n-propylthiourea, tricyclohexylthiourea, tetramethylthiourea, tetraethylthiourea, tetra-n-propylthiourea, and tetracyclohexylthiourea.

The chemical polymerization initiator may be a combination of the oxidant, the reductant, and an optionally added polymerization accelerator. Examples of the polymerization accelerator include aliphatic amines, aromatic tertiary amines containing an electron-withdrawing group, sulfinic acids and/or salts thereof, reducible inorganic compounds containing sulfur, reducible inorganic compounds containing nitrogen, borate compounds, barbituric acid derivatives, triazine compounds, copper compounds, tin compounds, vanadium compounds, halogen compounds, aldehydes, and thiol compounds.

The initiator can be a photoinitiator, such as, for example, one or more of a (bis)acylphosphine oxide, an alpha-diketone, and a coumarin.

Examples of the (bis)acylphosphine oxides, particularly acylphosphine oxides, which may be used as the photoinitiator include 2,4,6-trimethylbenzoyldiphenylphosphine oxide, 2,6-dimethoxybenzoyldiphenylphosphine oxide, 2,6-dichlorobenzoyldiphenylphosphine oxide, 2,4,6-trimethylbenzoylmethoxyphenylphosphineoxide,2,4,6-trimethylbenzoylethoxyphenylphosphine oxide, 2,3,5,6-tetramethylbenzoyldiphenylphosphine oxide, benzoyl di-(2,6-dimethylphenyl)phosphonate, (2,5,6-trimethylbenzoyl)-2,4,4-trimethylpentylphosphine oxide, and 2,4,6-trimethylbenzoylphenylphosphine oxide sodium salt. Examples of the bisacylphosphine oxides include bis(2,6-dichlorobenzoyl)phenylphosphine oxide, bis(2,6-dichlorobenzoyl)-2,5-dimethylphenylphosphine oxide, bis(2,6-dichlorobenzoyl)-4-propylphenylphosphine oxide, bis(2,6-dichlorobenzoyl)-1-naphthylphosphineoxide,bis(2,6-dimethoxybenzoy)phenylphosphine oxide, bis(2,6-dimethoxybenzoy)-2,4,4-trimethylpentylphosphine oxide, bis(2,6-dimethoxybenzoyl)-2,5-dimethylphenylphosphine oxide, and bis(2,4,6-trimethylbenzoy)phenylphosphineoxide.

Examples of alpha-diketones which may be used as photoinitiator include diacetyl, dibenzyl, camphorquinone, 2,3-pentadione, 2,3-octadione, 9,10-phenanthrenequinone, 4,4′-oxybenzyl, and acenaphthenequinone.

Examples of the coumarins which may be used as photoinitiator include 3,3′-carbonylbis(7-diethylamino)coumarin, 3-(4-methoxybenzoyl)coumarin, 3-thenoylcoumarin, 3-benzoyl-5,7-dimethoxycoumarin, 3-benzoyl-7-methoxycoumarin, 3-benzoyl-6-methoxycoumarin, 3-benzoyl-8-methoxycoumarin, 3-benzoylcoumarin, 7-methoxy-3-(p-nitrobenzoyl)coumarin, 3-(p-nitrobenzoyl)coumarin, 3,5-carbonylbis(7-methoxycoumarin), 3-benzoyl-6-bromocoumarin, 3,3′-carbonylbiscoumarin, 3-benzoyl-7-dimethylaminocoumarin, 3-benzoylbenzo[f]coumarin, 3-carboxycoumarin, 3-carboxy-7-methoxycoumarin, 3-ethoxycarbonyl-6-methoxycoumarin, 3-ethoxycarbonyl-8-methoxycoumarin, 3-acetylbenzo[f]coumarin,7-methoxy-3-(p-nitrobenzoyl)coumarin,3-(p-nitrobenzoyl)coumarin,3-benzoyl-6-nitrocoumarin, 3-benzoyl-7-diethylaminocoumarin, 7-dimethylamino-3-(4-methoxybenzoyl)coumarin, 7-diethylamino-3-(4-methoxybenzoyl)coumarin, 7-diethylamino-3-(4-diethylamino)coumarin, 7-methoxy-3-(4-methoxybenzoyl)coumarin, 3-(4-nitrobenzoyl)benzo[f]coumarin, 3-(4-ethoxycinnamoyl)-7-methoxycoumarin, 3-(4-dimethylaminocinnamoyl)coumarin, 3-(4-diphenylaminocinnamoyl)coumarin, 3-[(3-dimethylbenzothiazol-2-ylidene)acetyl]coumarin, 3-[(1-methylnaphto[1,2-d]thiazol-2-ylidene)acetyl]coumarin, 3,3′-carbonylbis(6-methoxycoumarin), 3,3′-carbonylbis(7-acetoxycoumarin), 3,3′-carbonylbis(7-dimethylaminocoumarin), 3-(2-benzothiazoyl)-7-(diethylamino)coumarin, 3-(2-benzothiazoyl)-7-(dibutylamino)coumarin, 3-(2-benzoimidazoyl)-7-(diethylamino)coumarin, 3-(2-benzothiazoyl)-7-(dioctylamino)coumarin, 3-acetyl-7-(dimethylamino)coumarin, 3,3′-carbonylbis(7-dibutylaminocoumarin), 3,3′-carbonyl-7-diethylaminocoumarin-7′-bis(butoxyethyl)aminocoumarin, 10-[3-[4-(dimethylamino)phenyl]-1-oxo-2-propenyl]-2,3,6,7-tetrahydro-1,1,-7,7-tetramethyl-1H,5H,11H-[1]benzopyrano[6,7,8-ij]quinolizin-11-one, and 10-(2-benzothiazoyl)-2,3,6,7-tetrahydro-1,1,7,7-tetramethyl-1H, 5H, 11H-[1]benzopyrano [6,7,8-ij]quinolizin-11-one.

Exemplary polymerization accelerator; that are usable in combination with a photoinitiator include tertiary amines, aldehydes, thiol group-containing compounds, and sulfinic acids and/or salts thereof.

In exemplary embodiments, a curable polymeric precursor formulation can further comprise a filler. Any of commonly-known inorganic particles used as a filler in dental composite resins can be used without any limitation. Specifically, for example, particles of the following conventionally-known materials can be used: various glass materials (containing silicon dioxide (quartz, quartz glass, silica gel, or the like) and silicon as main components and further containing heavy metal(s) and boron and/or aluminum); fluorine-containing glass materials such as fluoroaluminosilicate glass, calcium fluoroaluminosilicate glass, strontium fluoroaluminosilicate glass, barium fluoroaluminosilicate glass, and strontium calcium fluoroaluminosilicate glass; alumina; various ceramic materials; diatomite; kaolin; clay minerals (such as montmorillonite); activated white clay; synthetic zeolite; mica; silica; calcium fluoride; ytterbium fluoride; calcium phosphate; barium sulfate; zirconium dioxide (zirconia); titanium dioxide (titania); and hydroxyapatite.

When the curable formulation is configured for use as a dental adhesive, for example, for a tooth structure and a dental prosthesis, polymerizable materials featuring an acidic group are suitable.

Exemplary such materials include, for example, phosphate group-containing polymerizable materials such as 2-(meth)acryloyloxyethyl dihydrogen phosphate, 3-(meth)acryloyloxypropyl dihydrogen phosphate, 4-(meth)acryloyloxybutyl dihydrogen phosphate, 5-(meth)acryloyloxypentyl dihydrogen phosphate, 6-(meth)acryloyloxyhexyl dihydrogen phosphate, 7-(meth)acryloyloxyheptyl dihydrogen phosphate, 8-(meth)acryloyloxyoctyl dihydrogen phosphate, 9-(meth)acryloyloxynonyl dihydrogen phosphate, 10-(meth)acryloyloxydecyl dihydrogen phosphate, 11-(meth)acryloyloxyundecyl dihydrogen phosphate, 12-(meth)acryloyloxydodecyl dihydrogen phosphate, 16-(meth)acryloyloxyhexadecyl dihydrogen phosphate, 20-(meth)acryloyloxyeicosyl dihydrogen phosphate, bis[2-(meth)acryloyloxyethyl]hydrogenphosphate,bis[4-(meth)acryloyloxybutyl]hydrogenphosphate, bis[6-(meth)acryloyloxyhexyl]hydrogen phosphate, bis[8-(meth)acryloyloxyoctyl]hydrogen phosphate, bis[9-(meth)acryloyloxynonyl]hydrogen phosphate, bis[10-(meth)acryloyloxydecyl]hydrogen phosphate, 1,3-di(meth)acryloyloxypropyl-2-dihydrogen phosphate, 2-(meth)acryloyloxyethylphenylhydrogen phosphate, 2-(meth)acryloyloxyethyl-2′-bromoethyl hydrogen phosphate, 2-methacryloyloxyethyl(4-methoxyphenyl) hydrogen phosphate, 2-methacryloyloxypropyl(4-methoxyphenyl) hydrogen phosphate, glycerol phosphate di(meth)acrylate, and dipentaerythritol phosphate penta(meth)acrylate; and acid chlorides thereof; phosphonate group-containing polymerizable materials such as 2-(meth)acryloyloxyethylphenyl phosphonate, 5-(meth)acryloyloxypentyl-3-phosphonopropionate, 6-(meth)acryloyloxyhexyl-3-phosphonopropionate, 10-(meth)acryloyloxydecyl-3-phosphonopropionate, 6-(meth)acryloyloxyhexyl-3-phosphonoacetate, and 10-(meth)acryloyloxydecyl-3-phosphonoacetate, and acid chlorides thereof; pyrophosphate group-containing polymerizable materials such as bis[2-(meth)acryloyloxyethyl]pyrophosphate, bis[4-(meth)acryloyloxybutyl]pyrophosphate, bis[6-(meth)acryloyloxyhexyl] pyrophosphate, bis[8-(meth)acryloyloxyoctyl]pyrophosphate,andbis[10-(meth)acryloyloxydecyl]pyrophosphate; and acid chlorides thereof; carboxylate group-containing polymerizable materials such as maleic acid, methacrylic acid, 4-[2-[(meth)acryloyloxy]ethoxycarbonyl]phthalic acid, 4-(meth) acryloyloxybutyloxycarbonylphthalicacid,4-(meth)acryloyloxyhexyloxycarbonylphthalicacid, 4-(meth) acryloyloxyoctyloxycarbonylphthalic acid, 4-(meth) acryloyloxydecyloxycarbonylphthalic acid, acid anhydrides thereof, 5-(meth) acryloylaminopentylcarboxylic acid, 6-(meth)acryloyloxy-1,1-hexanedicarboxylic acid, 8-(meth)acryloyloxy-1,1-octanedicarboxylic acid, 10-(meth)acryloyloxy-1,1-decanedicarboxylic acid, and 11-(meth)acryloyloxy-1,1-undecanedicarboxylic acid; and acid chlorides thereof; sulfonate group-containing polymerizable materials such as 2-(meth)acrylamido-2-methylpropanesulfonic acid, styrenesulfonic acid, and 2-sulfoethyl (meth)acrylate; and acid chlorides thereof; thiophosphate group-containing polymerizable materials such as 10-(meth)acryloyloxydecyl dihydrogen dithiophosphate; and acid chlorides thereof.

When the dental curable composition is used as a glass ionomer cement, it typically includes a curable material such as a polyalkenoic acid an ion-leachable glass, and water. The polyalkenoic acid can be a (co)polymer of an unsaturated carboxylic acid such as an unsaturated monocarboxylic acid or an unsaturated dicarboxylic acid, and examples of the (co)polymer include homopolymers of acrylic acid, methacrylic acid, 2-chloroacrylic acid, 2-cyanoacrylic acid, aconitic acid, mesaconic acid, maleic acid, itaconic acid, fumaric acid, glutaconic acid, citraconic acid, and the like; copolymers of two or more of these unsaturated carboxylic acids; and copolymers of these unsaturated carboxylic acids with other monomers copolymerizable with the unsaturated carboxylic acids. These polymers may be used alone or two or more thereof may be used in combination. In the case of a copolymer of any of the unsaturated carboxylic acids with another copolymerizable monomer, the proportion of the unsaturated carboxylic acid unit is preferably 50 mol % or more in the total structural units. The copolymerizable monomer is preferably an ethylenically unsaturated polymerizable monomer, and examples thereof include styrene, acrylamide, acrylonitrile, methyl methacrylate, acrylic acid salts, vinyl chloride, allyl chloride, vinyl acetate, and 1,1,6-trimethylhexamethylene dimethacrylate. Among these polyalkenoic acids, at least one selected from the group consisting of homopolymers of acrylic acid, maleic acid, and itaconic acid, a copolymer of acrylic acid with maleic acid, and a copolymer of acrylic acid with itaconic acid is preferable, and the copolymer of acrylic acid with itaconic acid is particularly preferable, in terms of improvement in bond strength to a tooth structure and in mechanical strength.

Examples of ion-leachable glass include fluoroaluminosilicate glass, calcium fluoroaluminosilicate glass, strontium fluoroaluminosilicate glass, barium fluoroaluminosilicate glass, and strontium calcium fluoroaluminosilicate glass.

Dental curable composition usable as a resin-modified glass ionomer cement typically include any of the polymerizable materials as described hereinabove, a polymerization initiator, a polyalkenoic acid, an ion-leachable glass and water, each as described hereinabove.

Each of the dental formulations described herein can further include one or more water-soluble fluoride compound or fluorine-releasing polymer. Examples of the water-soluble fluoride compound include water-soluble metal fluorides such as lithium fluoride, sodium fluoride, potassium fluoride, rubidium fluoride, cesium fluoride, beryllium fluoride, magnesium fluoride, calcium fluoride, strontium fluoride, barium fluoride, zinc fluoride, aluminum fluoride, manganese fluoride, copper fluoride, lead fluoride, silver fluoride, antimony fluoride, cobalt fluoride, bismuth fluoride, tin fluoride, diammine silver fluoride, sodium monofluorophosphate, potassium fluorotitanate, fluorostannate, and fluorosilicate.

Each of the dental formulations described herein can further include can further contain an inorganic calcium compound. Examples of the inorganic calcium compound include acidic calcium phosphate particles, basic calcium phosphate particles, and calcium compounds containing no phosphorus.

The dental formulation may further contain a stabilizer (polymerization inhibitor), a colorant, a fluorescent agent, and an ultraviolet absorber.

A composition as described herein can further comprise one or more pharmaceutically active agents (in addition to the nanostructures and the polymeric precursor).

Non-limiting examples of pharmaceutically active ingredients include Analgesics, Antibiotics, Anticoagulants, Antidepressants, Anticancers, Antiepileptics, Antipsychotics, Antivirals, Sedatives and Antidiabetics. Non-limiting examples of Analgesics include paracetamol, non-steroidal anti-inflammatory drugs (NSAIDs), morphine and oxycodone. Non-limiting examples of Antibiotics include penicillin, cephalosporin, ciprofolxacin and erythromycin. Non-limiting examples of Anticoagulants include warfarin, dabigatran, apixaban and rivaroxaban. Non-limiting examples of Antidepressants include sertraline, fluoxetine, citalopram and paroxetine. Non-limiting examples of Anticancers include Capecitabine, Mitomycin, Etoposide and Pembrolizumab. Non-limiting examples of Antiepileptics include Acetazolamide, Clobazam, Ethosuximide and lacosamide. Non-limiting examples of Antipsychotics include Risperidone, Ziprasidone, Paliperidone and Lurasidone. Non-limiting examples of Antivirals include amantadine, rimantadine, oseltamivir and zanamivir. Non-limiting examples of Sedatives include Alprazolam, Clorazepate, Diazepam and Estazolam. Non-limiting examples of Antidiabetics include glimepiride, gliclazide, glyburide and glipizide.

The composition may further comprise excipients, such as, but not limited to, binders, coatings, lubricants, flavors, preservatives, sweeteners, vehicles and disintegrants. Non-limiting examples of binders include saccharides, gelatin, polyvinylpyrolidone (PVP) and polyethylene glycol (PEG). Non-limiting examples of coatings include hydroxypropylmethylcellulose, polysaccharides and gelatin. Non-limiting examples of lubricants include talc, stearin, silica and magnesium stearate. Non-limiting examples of disintegrants include crosslinked polyvinylpyrolidone, crosslinked sodium carboxymethyl cellulose (croscarmellose sodium) and modified starch sodium starch glycolate.

Any other excipients suitable for administration into an oral cavity are contemplated.

According to some of any of the embodiments described herein, a weight ratio of the plurality of nanostructures and the polymeric precursor formulation ranges from 1:1000 to 1:10, or from 1:100 to 1:10, or from 1:100 to 1:20, or from 1:100 to 1:50.

According to some of any of the embodiments described herein, a concentration of the plurality of nanostructures in the polymeric precursor mixture ranges from about 0.1% to 10%, or from 0.1% to 5%, or from 0.1% to 2%, or from 1% to 5%, or from 1% to 3%, or from 1% to 2%, by weight, including any intermediate values and subranges therebetween.

Process:

According to an aspect of some embodiments of the present invention there is provided a process of preparing the composition described herein.

In some embodiments, the process comprises mixing the plurality of the nanostructures and the dental formulation as described herein in any of the respective embodiments.

In some embodiments, the dental formulation is a curable formulation as described herein and the process comprises mixing the plurality of the nanostructures and the polymeric precursor formulation as described herein in any of the respective embodiments.

In some of any of the embodiments described herein, the mixing comprises repetitively subjecting a mixture of the nanostructures and the polymeric precursor formulation to manual mixing, centrifugation and/or sonication. In some embodiments, the mixing comprises manual mixing, centrifugation and sonication. In some embodiments, the mixing comprises repetitive manual mixings (e.g., 3-10 times), followed by or interrupted by repetitive centrifugation (e.g., 2-5 times), followed by or interrupted by sonication. An exemplary mixing procedure is described in the Examples section that follows.

In some embodiments, the plurality of the nanostructures and the dental formulation (e.g., dental curable formulation) are mixed at a weight ratio as described herein in the respective embodiments.

In some embodiments, the process further comprises, prior to the mixing, forming the plurality of nanostructures, the forming comprising diluting a solution comprising the aromatic molecules and an organic solvent with an aqueous solution. In some embodiments, the organic solvent is a polar organic solvent, and in some embodiments it is a protic polar organic solvent, for example, an alcohol such as ethanol. In some embodiments, the dilution is to a final concentration that ranges from 1 mg/ml to 20 mg/ml or from 1 mg/ml to 10 mg/ml, including any intermediate values and subranges therebetween.

Composite Material:

The compositions described herein, which comprise a curable formulation, can form a composite material that comprises a polymeric matrix that is usable in a dental application (e.g., a dental composite restorative as described herein), for example, upon being applied to an area in an oral cavity of a subject in need thereof, the polymeric matrix having dispersed therein nanostructures as described herein.

According to an aspect of some embodiments of the present invention the composition as described herein, which comprises a curable dental formulation, is usable for forming such a polymeric matrix.

According to an aspect of some embodiments of the present invention the composition as described herein, which comprises a curable dental formulation, is usable for forming a dental restorative material. According to an aspect of some embodiments of the present invention there is provided a polymeric matrix usable in dental application and a plurality of self-assembled nanostructures dispersed within the polymeric matrix.

In some embodiments, the polymeric matrix is such that is usable in a dental application as described herein.

In some embodiments, the plurality of nanostructures comprises nanostructures formed of a plurality of aromatic molecules, each of the aromatic molecules comprising an aromatic amino acid, as described herein in any of the respective embodiments.

In some embodiments, the composite material is prepared upon subjecting the composition which comprises a curable dental formulation, as described herein in any of the respective embodiments, to conditions under which the polymeric matrix is formed of the polymeric precursor formulation.

In some embodiments, the composite material is prepared upon subjecting the composition as described herein in any of the respective embodiments, which comprises a curable dental formulation, to conditions for effecting polymerization and/or curing of the polymeric precursor formulation.

According to some of any of the embodiments described herein, the plurality of nanostructures in the composite material is as described herein in any of the respective embodiments and any combination thereof.

According to some of any of the embodiments described herein, the polymeric matrix is obtainable from a polymeric precursor mixture or formulation as described herein in any of the respective embodiments (e.g., upon subjecting the mixture to a suitable condition).

According to some of any of the embodiments described herein, the composition comprises the nanostructure as described herein and a polymeric material which can comprise organic polymers, inorganic polymers or any combination thereof.

In some embodiments, the nanostructure(s) as described herein are dispersed in the polymeric material. In some embodiments, the nanostructure(s) are homogeneously dispersed within the polymeric material. In some embodiments, the nanostructure(s) are found in the surface of the polymeric material. In some embodiments, the nanostructure(s) coat the polymeric material. In some embodiments, the nanostructure(s) interact weakly or physically (mechanically) with the polymeric material. In some embodiments, the nanostructure(s) are mechanically embedded within the polymeric material. In some embodiments, the nanostructure(s) are three dimensionally “locked” between the polymer chains, preventing them from migrating out from the complex network. In some embodiments, the polymeric material is inert to the nanostructure(s) and does not react chemically with the nanostructure(s).

Any polymeric materials and/or matrices formed of polymeric precursors as described herein is encompassed by these embodiments.

In some of any of the embodiments described herein, the composite material is usable as, or as a part of, a medical device or composite for dental appliance and/or for orthodontic appliance and/or for periodontal appliance.

In some of any of the embodiments described herein, the composite material is, or forms a part of, medical devices or composite such as, but not limited to, a dental adhesive, a bone cement, a dental restorative material such as materials for filling tooth-decay cavities, endodontic filling materials (cements and fillers) for filling the root canal space in root canal treatment, materials used for provisional and final tooth restorations or tooth replacement, including but not restricted to inlays, onlays, crowns, partial dentures (fixed or removable) dental implants, and permanent and temporary cements used in dentistry for various known purposes, dental resin based cements, dental sealers, and a varnish or glaze which is applied to the tooth surface, a restoration of tooth or a crown, for example, for sealing open pores in the surface of a fired porcelain.

In some of any of the embodiments described herein, the composite material is, or forms a part of, medical devices or composite such as, but not limited to, an aligner for accelerating the tooth aligning, a bracket, a dental attachment, a bracket auxiliary, a ligature tie, a pin, a bracket slot cap, a wire, a screw, a micro-staple, cements for bracket and attachments and other orthodontic appliances, a denture, a partial denture, a dental implant, a periodontal probe, a periodontal chip, a film, or a space between teeth, a mouth guard, used to prevent tooth grinding (bruxer, Bruxism), night guard, an oral device used for treatment/prevention sleep apnea, teeth guard used in sport activities.

According to some of any of the embodiments described herein, the composite material is a dental restorative filling material, a dental adhesive material, a dental luting material, a dental temporary sealing material, a dental provisional crown material, or a dental pit and fissure sealant, or any other composite usable for dental, orthodental or periodontal treatment or appliance, according to methods known to those skilled in the art.

The polymeric matrix in the composite material is formed of any of the polymeric precursors and formulations thereof, as described herein in any of the respective embodiments.

Dental adhesive materials can be used as a restorative material for a tooth structure, or for a crown restoration material (made of metal, porcelain, ceramic, cured composite, or the like) fractured in an oral cavity.

According to some embodiments, the composite material is for use as a dental restorative composite.

According to some of any of the embodiments described herein, the composite material features at least one mechanical and/or optical property that is substantially similar to that of the polymeric matrix without the nanostructure(s). Such a property can be, for example, toughness, stiffness, tensile strength, hardness, color, or any other spectral or optical property (e.g., refractive index).

According to some of these embodiments, one or more of these properties differs from the same property as measured for the same polymeric matrix without the nanostructures by no more than 20%, or no more than 15%, or no more than 10%, or not more than 5 5, or no more than 3%, or no more than 1%.

According to some of any of the embodiments described herein, a toughness of the composite material differs from a toughness of the same polymeric matrix without the nanostructures by no more than 20%, preferably by no more than 15%, no more than 10%, or less. In some embodiments, the relative toughness is determined statistically by Dunnett post hoc test comparing the composite material with a control native material (without nanostructures).

According to some of any of the embodiments described herein, a stiffness of the composite material differs from a stiffness of the same polymeric matrix without the nanostructures by no more than 20%, preferably by no more than 15%, no more than 10%, or less.

According to some of any of the embodiments described herein, a tensile strength of the composite material differs from a tensile strength of the same polymeric matrix without the nanostructures by no more than 20%, preferably by no more than 15%, no more than 10%, or less.

According to some of any of the embodiments described herein, a refractive index of the composite material differs from a toughness of the same polymeric matrix without the nanostructures by no more than 20%, preferably by no more than 15%, no more than 10%, or less.

According to some of any of the embodiments described herein, a color or any other spectral property of the composite material differs from a toughness of the same polymeric matrix without the nanostructures by no more than 20%, preferably by no more than 15%, no more than 10%, or less.

The relative property can be determined by comparing the composite material with a control native material (without nanostructures), using methods and assays well known and widely practiced in the art. Exemplary such methods are described in the Examples section that follows.

According to some of any of the embodiments described herein, the composite material is characterized as non-toxic to eukaryotic cells and hence as biocompatible and suitable for application in an oral cavity of a subject in need thereof. Reference is made in this regard, for example, to FIGS. 6A-D and accompanying description.

According to some of any of the embodiments described herein, the composite material is characterized as featuring an antimicrobial activity, for example, an anti-bacterial activity, as described herein.

According to some of the any of the embodiments described herein, the composite material as described herein is usable, or is for use, in reducing a load of a microorganism (e.g., bacteria) in and/or on a substrate, such as an organ or a tissue in the oral cavity (e.g., a tooth, a gum).

Reducing a load of a microorganism (e.g., bacteria) is by at least 50%, or at least 60%, or at least 80%, or by higher, and can be inhibiting growth, reduction in the growth rate of the bacteria; reduction in the size of the population of the bacteria; prevention of growth of the bacteria; causing irreparable damage to the bacteria; destruction of a biofilm of such bacteria; inducing damage, short term or long term, to a part or a whole existing biofilm; preventing formation of such biofilm; inducing biofilm management; or bringing about any other type of consequence which may affect such population or biofilm and impose thereto an immediate or long term damage (partial or complete).

In some of any of the embodiments described herein, a composite material as described herein, is usable, or is for use, in inhibiting, reducing or preventing biofilm formation on a substrate, for example, an organ or tissue in an oral cavity.

According to some of any of the embodiments described herein there is provided a method of inhibiting bacteria, as described herein, and/or of inhibiting or preventing biofilm formation, in and/or on a substrate (e.g., an organ or tissue in the oral cavity), which comprises contacting the substrate with a composite material as described herein in any of the respective embodiments.

According to an aspect of some embodiments of the present invention there is provided a method of treating a dental and/or periodontal infection, or any other dental, orthodental or periodontal condition in which antibacterial or anti-biofilm formation activity is beneficial, which comprises contacting an infected area in the oral cavity of a subject in need thereof with a composition as described herein in any of the respective embodiments.

According to an aspect of some embodiments of the present invention there is provided a composition as described herein in any of the respective embodiments, for use in treating or preventing a dental and/or periodontal infection, or any other dental, orthodental or periodontal condition in which antibacterial or anti biofilm formation activity is beneficial, According to some embodiments, the infection is associated with formation of dental plaque, as described herein and in the art.

According to some embodiments, the dental, orthodental or periodontal condition is such that is treated by a composite material as described herein in any of the respective embodiments.

According to some embodiments of the present invention there is provided a method of treating or preventing a dental and/or periodontal infection, which is effected by contacting an infected area in the oral cavity of a subject in need thereof with a dental composition or with a composite material as described herein in any of the respective embodiments.

According to some embodiments of the present invention there is provided a method of method of treating a dental, periodontal or orthodontic condition in which treating or preventing a bacterial infection and/or reducing, inhibiting or retarding biofilm formation is beneficial (e.g., a condition associate with dental plaque) in a subject in need thereof, which is effected by contacting an organ or a tissue in the oral cavity of the subject with a composition or composite material according to any of the respective embodiments.

According to some embodiments of the present invention there is provided a dental composition or dental composite as described herein in any of the respective embodiments, for use in inhibiting, reducing or retarding a formation of a bacterial load in the oral cavity (e.g., in a tissue or organ in the oral cavity).

Compositions and Articles of manufacturing:

Embodiments of the present invention further relate to utilizing the antibacterial and ABF activities of self-assembled nanostructures made of halogenated aromatic amino acids as described herein in any of the respective embodiments (e.g., nanostructures made of self-assembled pentafluorophenylalanine, preferably modified at the alpha amine by an aromatic end-capping moiety), in applications in which such an activity is beneficial.

According to some embodiments of the present invention there is provided a composition comprising at least one nanostructure formed of self-assembled plurality of aromatic molecules, wherein each of said aromatic molecules comprises a halogenated aromatic amino acid, as described herein, which is for use in inhibiting, reducing or retarding a formation of a bacterial load in and/or a substrate.

According to some embodiments of the present invention there is provided a method of inhibiting, reducing or retarding a formation of a bacterial load in and/or a substrate, which is effected by contacting the substrate with a composition comprising at least one nanostructure formed of self-assembled plurality of aromatic molecules, wherein each of said aromatic molecules comprises a halogenated aromatic amino acid, as described herein.

Embodiments of the present invention further encompass articles-of-manufacturing comprising a composition as described herein, and some embodiments are of articles-of-manufacturing that are made of polymeric materials, for example, as described herein for dental composites or formulations, which can be prepared upon mixing the nanostructures with a polymeric precursor formulation usable for forming the polymeric articles.

According to some embodiments of the present invention there is provided an article-of-manufacture comprising a polymeric matrix and the composition as described in these embodiments incorporated in and/or the polymeric matrix.

Exemplary polymeric precursors also include precursors of organic polymers, inorganic polymers and a combination thereof. Exemplary precursors include precursors of thermoplastic polymers, thermoset polymers or any combination thereof. Precursors of organic polymers may include precursors of hydrogels, polyolefins such as polyvinylchloride (PVC), polyethylene, polystyrene and polypropylene, of epoxy resins, of acrylate resins such as poly methyl methacrylate, polyurethane or any combination thereof. Precursors of inorganic polymers include, for example, precursors of silicone polymers such as polydimethylsiloxane (PDMS), ceramics, metals or any combination thereof. Exemplary hydrogels include poloxamers or alginates.

Exemplary polymeric matrices are those formed of the described polymeric precursors. Also contemplated are polymeric matrices described herein for dental composite materials.

In some embodiments, term “reducing the load” refers to a decrease in the number of the microorganism(s), e.g., bacteria, or bacterial biofilm, or to a decrease in the rate of their growth or formation, or both in the substrate as compared to a non-treated substrate.

A substrate as defined herein throughout encompasses living tissues (animate) and inanimate substrates or objects.

In the context of embodiments of the present invention, the phrase “living tissue” is meant to encompass any part of a living organism, a bodily site or a living organ.

As used herein, the phrase “bodily site” includes any organ, tissue, membrane, cavity, blood vessel, tract, biological surface or muscle, which contacting therewith (e.g., delivering thereto or applying thereon) the composition disclosed herein is beneficial. Exemplary bodily sites include, but are not limited to, the skin, a dermal layer, the scalp, an eye, an ear, a mouth, a throat, a stomach, a small intestines tissue, a large intestines tissue, a kidney, a pancreas, a liver, the digestive system, the respiratory tract, a bone marrow tissue, a mucosal membrane, a nasal membrane, the blood system, a blood vessel, a muscle, a pulmonary cavity, an artery, a vein, a capillary, a heart, a heart cavity, a male or female reproductive organ and any visceral organ or cavity. Any organ or tissue onto or in which microorganism such as bacteria, or a biofilm, can exist or form in contemplated.

The phrase “living tissue” encompasses also samples of a living organism or subject, namely a human or an animal, which have been removed from the organism and maintained viable for any purpose, and encompasses the living subject itself as a whole, e.g., a plant, a human or an animal (e.g., a mammal).

In the context of embodiments of the present invention, the phrase “inanimate object” is meant to encompass any surface of an object which may harbor a microorganism, such as, but not limited to, an implantable medical device such as a gastric or duodenal sleeve, a topical medical device such as a wound dressing, a subcutaneous medical device such as a subcutaneous injection port, a percutaneous medical device such as a catheter, a syringe needle or an endoscopic device, a vessel, a tube, a lid, a wrap, a package, a work surface or area, a warehouse, a package and the like, as is further described hereinafter in the context of “substrate”.

As used herein, the phrase “inhibiting the growth” refers to an effect of a composition which stops and/or reverses the propagation of a microorganism, such that at least one cell or a culture thereof is no longer multiplying or growing and/or is killed as a result of coming in contact with the composition or composite.

According to some embodiments of the present invention, a composition as described herein is packaged in a packaging material and is identified in print, in or on the packaging material for use as an antibacterial or ABF composition to be applied in and/or on inanimate objects, as discussed herein. Such a composition may be in a form of, for example, solution, paste, liquid, spray or powder.

According to some embodiments of this aspect of the present invention, when the substrate is a living tissue, the composition is a pharmaceutical composition.

Hence, according to an aspect of some embodiments of the present invention, there is provided a pharmaceutical composition which comprises nanostructure as described in the context of these embodiments and a pharmaceutically acceptable carrier.

Hereinafter, the term “pharmaceutically acceptable carrier” refers to a carrier or a diluent that does not cause significant irritation to an organism and does not inhibit the distribution, therapeutic properties or otherwise does not abrogate the biological activity and properties of the administered or applied compound.

Herein the term “excipient” refers to an inert substance added to a pharmaceutical composition to further facilitate administration or application of a drug.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the active ingredient(s) into preparations which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p.1).

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including transdermally, ophtalmically, vaginally, rectally, intranasally).

In some embodiments, the pharmaceutical composition is formulated as a wound dressing, using methods known in the art.

The amount of a composition to be administered or otherwise applied will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising active ingredient(s) according to embodiments of the invention formulated in a compatible pharmaceutical carrier may also be prepared, placed in an appropriate container, and labeled for treatment of a particular medical condition, disease or disorder, as is detailed herein.

According to some embodiments, the pharmaceutical composition presented herein is packaged in a packaging material and identified in print, in or on the packaging material, for use in inhibiting a growth of a pathogenic microorganism (e.g., bacteria) in a subject in need thereof.

When the substrate is a living tissue as defined herein, the product is a medicament. In the context of some embodiments of the present invention, the term “medicament” is used interchangeably with the phrase “pharmaceutical composition”.

When the substrate is an inanimate object as defined herein, the product is also referred to herein as an article or article-of-manufacture.

The article-of-manufacture according to some of the present embodiments, can be, for example, a biosensor, a medicament, a drug delivery system, a cosmetic or cosmeceutical agent, an implant, an artificial body part, a tissue engineering and regeneration system, and a wound dressing, as well as other various medical devices.

The article-of-manufacture according to some of the present embodiments, can alternatively be a packaging material, for example, a food packaging material or a packaging material of medical devices, drugs, cosmetic products, beverages, and the like.

As used herein, the term “implant” refers to artificial devices or tissues which are made to replace and act as missing biological structures. These include, for example, dental implants, artificial body parts such as artificial blood vessels or nerve tissues, bone implants, and the like.

As used herein, the phrase “tissue engineering and regeneration” refers to the engineering and regeneration of new living tissues in vitro, which are widely used to replace diseased, traumatized or other unhealthy tissues.

As used herein, the phrase “cosmetic or cosmeceutical agent” refers to topical substances that are utilized for aesthetical purposes. Cosmeceutical agents typically include substances that further exhibit therapeutic activity so as to provide the desired aesthetical effect. Cosmetic or cosmeceutical agents in which the hydrogels, compositions-of-matter and compositions described herein can be beneficially utilized include, for example, agents for firming a defected skin or nail, make ups, gels, lacquers, eye shadows, lip glosses, lipsticks, and the like.

Medical devices in which the hydrogels, compositions-of-matter and compositions described herein can be beneficially utilized include, for example, anastomotic devices (e.g., stents), sleeves, films, adhesives, scaffolds and coatings.

Anastomosis is the surgical joining of two organs. It most commonly refers to a connection which is created between tubular organs, such as blood vessels (i.e., vascular anastomosis) or loops of intestine. Vascular anastomosis is commonly practiced in coronary artery bypass graft surgery (CABG), a surgical procedure which restores blood flow to ischemic heart muscle in which blood supply has been compromised by occlusion or stenosis of one or more of the coronary arteries.

Stents can be used, for example, as scaffolds for intraluminal end to end anastomoses; as gastrointestinal anastomoses; in vascular surgery; in transplantations (heart, kidneys, pancreas, lungs); in pulmonary airways (trachea, lungs etc.); in laser bonding (replacing sutures, clips and glues) and as supporting stents for keeping body orifices open.

Sleeves can be used, for example, as outside scaffolds for nerves and tendon anastomoses.

Films can be used, for example, as wound dressing, substrates for cell culturing and as abdominal wall surgical reinforcement.

Coatings of medical devices can be used to render the device biocompatible, having a therapeutic activity, a diagnostic activity, and the like.

Other devices include, for example, catheters, aortic aneurysm graft devices, a heart valve, indwelling arterial catheters, indwelling venous catheters, needles, threads, tubes, vascular clips, vascular sheaths and drug delivery ports, which can be made of polymeric material incorporating the nanostructures or be coated with such a polymeric film.

Herein throughout, in some embodiments, the phrase “pathogenic microorganism” refers to a bacterium (or a bacterial strain).

The terms “bacterium” or “bacteria”, as used herein, refers to all prokaryotic organisms, including those within all of the phyla in the Kingdom Procaryotae. It is intended that these terms encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within these terms are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram stain, causing the stained cells to generally appear dark blue to purple under the microscope. “Gram-negative bacteria” do not retain the primary dye used in the Gram stain, but are stained by the counterstain. Thus, Gram-negative bacteria generally appear red. In some embodiments, bacteria are continuously cultured. In some embodiments, bacteria are uncultured and existing in their natural environment (e.g., at the site of a wound or infection) or obtained from patient tissues (e.g., via a biopsy). Bacteria may exhibit pathological growth or proliferation. Non-limiting examples of bacteria include bacteria of a genus selected from the group including Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plessiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolysporas, The rmoactinomycetes, Mycobacterium, Treponema, Borrelia, Leptospira and Chlamydiae.

In some embodiments of the present invention the pathogenic bacteria are of one or more of the following species: Acinetobacter baumanii, Belicobacter pylori, Burkholderia multivorans, Canipylobacter jejuni, Deinococcus radiodurans, E. coli, Enterobacter cloacae, Enterococcus faecalis, Haemophilus influenzae, Klebsiella pneumoniae, Klebsiella oxytoca, Mycobacterium tuberculosis, Neisseria gonorrhoeae, Neisseria meningitidis, Pseudomonas aeruginosa, Pseudomonas phosphoreui, Escherichia coli, Bacillus Subtifis, Borrelia burgfrferi, N'isseria meningitidis, N'isseria gonorrhocae, Yersinia pestis, Canipylobacter jejuni, Deinococcus radiodurans, Mycobacterium tuberculosis, Enterococeus faecalis, Streptococcus pneumoniae, Streptococcus pyogenes and Staphylococcus aureus, Salmonella typhimuriunim, Serratia marcescens, Staphylococcus aureus, Staphylococcus epidermidis, Staphylococcus pneumoniae, Staphylococcus sanguis, Staphylococcus viridans, Vibrio harveyi, Vibrio cholerae, Vibrio parahaeniolyticus, Vibrio alginolyticus, Yersinia enterocolitica or Yersinia pestis, including any strain or mutant thereof.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “alkyl” refers to a saturated aliphatic hydrocarbon including straight chain and branched chain groups. Preferably, the alkyl group has 1 to 20 carbon atoms. The alkyl group may be substituted or unsubstituted. When substituted, the substituent group can be, for example, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine. Representative examples are piperidine, piperazine, tetrahydro furane, tetrahydropyrane, morpholino and the like.

A “hydroxy” group refers to an —OH group.

A “thio”, “thiol” or “thiohydroxy” group refers to and —SH group.

An “azide” group refers to a —N═N≡N group.

An “alkoxy” group refers to both an —O-alkyl and an —O-cycloalkyl group, as defined herein.

An “aryloxy” group refers to both an —O-aryl and an —O-heteroaryl group, as defined herein. A “thiohydroxy” group refers to and —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “halo” or “halide” group refers to fluorine, chlorine, bromine or iodine. A “trihaloalkyl” group refers to an alkyl substituted by three halo groups, as defined herein.

A representative example is trihalomethyl.

An “amino” group refers to an —NR′R″ group where R′ and R″ are hydrogen, alkyl, cycloalkyl or aryl.

A “nitro” group refers to an —NO₂ group.

A “cyano” group refers to a —CN group.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### MATERIALS AND EXPERIMENTAL METHODS

Materials: Lyophilized powders of Fmoc-Pentafluorophenylalanline (Fmoc-Pentafluoro-Phe; Fmoc-F5-Phe) and Pentafluorophenylalanine (Pentafluoro-Phe; H-F5-Phe) were purchased from Chem-Impex INT'L inc.

Fmoc-Phe was purchased from Sigma Aldrich (Rehovot, Israel).

All powders were used without further purification.

Filtek Ultimate Flow dental resin composite restorative (3M, ESPE) was used.

Nanostructure Formation: Nanostructures were prepared by the solvent switch method, according to procedures described, for example, in Mahler et al. Adv. Mater., 18(11), 1365-1370; and Halperin-Sternfeld et al. Chem. Commun., 53(69), 9586-9589. First, stock solutions of each peptide powder was prepared in ethanol, and then diluted with DDW. In case of Fmoc-Phe and H-F5-Phe, the stock solution was 20 mg/ml in ethanol and the final concentration was 2 mg/ml in DDW with 10% ethanol (90% of DDW and 10% ethanol). In case of Fmoc-F5-Phe the stock solution was 10 mg/ml in ethanol and the final concentration was 1 mg/ml in DDW with 10% ethanol (90% of DDW and 10% ethanol). Immediately after dilution, the resulting solutions were strongly mixed by vortex and placed aside undisturbed to permit self-assembling processes. The formed nanostructures were lyophilized overnight, with the resulting ethanol concentration in these samples being substantially zero due to the lyophilization process.

Scanning Electron Microscopy (SEM): Samples of the formed nanostructures were deposited on carbon tape and micrographs were recorded using a JEOL JSM-6700F FE-SEM scanning electron microscope operating at 10 KV. Micrographs displayed are representative of three independent experiments conducted.

Energy-Dispersive X-ray Spectroscopy (EDX): Nanostructures-containing dental restorative composites were light-cured on glass slides and imaged using a JSM-6700F FEG-SEM (JEOL, Tokyo, Japan). EDX analysis using Oxford INCA (Oxford Instruments America, Inc., Concord, Mass.) was carried out on the visualized sample area.

Other methods are described in detail in the following.

### Example 1

### Preparation and Characterization of Peptide Nanostructures

Composite antimicrobial dental materials were prepared by incorporation of antimicrobial peptide nanostructures as disclosed herein within dental resin composite restoratives.

Nanostructures made of Fmoc-Pentafluorophenylalanline (Fmoc-Pentafluoro-Phe; Fmoc-F5-Phe), Fmoc-Phe and Pentafluorophenylalanine (Pentafluoro-Phe; H-F5-Phe) were prepared using the solvent-switch methodology as described hereinabove.

The formed nanostructures were lyophilized overnight. FIGS. 1A-C present micrographs obtained by Scanning Electron Microscopy (SEM) of the formed lyophilized nanostructures. As shown therein, the structures formed by Fmoc-F5-Phe are unbranched and elongated (fibrillary); they are in the ten-nanometer range (e.g., 25 nm) in terms of width and hundreds of nanometers range in terms of length. The structures formed by Fmoc-Phe and H—F₅-Phe are not as uniform and are in the 500 nm range in terms of width and micrometer range in terms of length. The structures formed by H-F5-Phe are the largest of the group and the least uniform, they seem to clump together to form wide flattened sheets. FIG. 1D presents micrographs of structures formed by Fmoc-F5-Phe, obtained using transmission electron microscopy (scale bar=2 μm).

The antibacterial capabilities of the obtained nanostructures were evaluated against S. mutans via a minimum inhibitory concentration (MIC) analysis and by kinetic growth inhibition analysis, as follows.

S. mutans bacteria were grown under anaerobic conditions in brain heart infusion (BHI) medium (BD Difco) for 48 hours and then diluted to OD₆₀₀ of 0.01 or 0.25 in BHI. Nanostructures samples, at an initial concentration of 8 mM, were added to the bacterial samples in 96-well plates in serial 2-fold dilutions, which were sealed to ensure anaerobic conditions. Kinetic growth inhibition was determined by optical density measurements (650 nm) using a Biotek Synergy HT microplate reader. The minimum inhibitory concentration (MIC) was determined using the microdilution assay, and evaluation of the reduction in colony-forming units was obtained by plating and counting bacterial samples before and after overnight treatment. The MIC was considered the lowest peptide concentration that showed no increase in optical density and no colony forming unit (CFU) growth overnight.

As shown in FIG. 1E, Fmoc-F5-Phe nanostructures exhibited substantial activity toward S. mutans as overnight incubation at 2 mM with cultures that started out at early log phase completely inhibited bacterial growth, with a reduction of 7.2 log(10) CFU/mL, with lower concentrations partially inhibiting growth in a dose-dependent manner. Presented kinetic analysis and MIC results are representative of three independent experiments conducted in quadruplets.

To directly assess bacterial viability, the bacteria were subjected to Live/Dead viability analysis containing Syto9 (indicating live bacteria) and Propidium iodide (PI) (indicating dead bacteria).

Following kinetic analysis, the samples were washed thrice with saline, incubated for 15 minutes in a solution containing Syto9 and Propidium iodide (L13152 LIVE/DEAD BacLight Bacterial Viability Kit, Molecular Probes, OR), and washed with saline again. Fluorescence emission was detected using an ECLIPSE E600 fluorescent microscope (Nikon, Japan).

The obtained data is presented in FIG. 1F and show that treatment with the Fmoc-F5-Phe nanostructures caused significant bacterial cell death. The presented results are representative of three independent experiments.

The ability of the nanostructures to inhibit bacterial growth at a higher bacterial load, similar to that of an active infection was also tested. S. mutans cultures were grown until mid-log phase and were then treated with 2 mM samples of the nanostructures.

The results are presented in FIGS. 1G and 1H. Kinetic growth inhibition analysis coupled with Live/Dead viability analysis revealed that the Fmoc-F5-Phe nanostructures inhibited bacterial proliferation at these concentrations.

The effect of Fmoc-F5-Phe nanostructures on bacterial morphology was studied using High-Resolution Scanning Electron Microscopy as follows.

Bacterial samples were centrifuged at 5000 rpm for 5 minutes, washed thrice in PBS, and fixed in 2.5% glutaraldehyde in PBS for 1 hour. The samples were then washed thrice in PBS and fixed in 1% OsO₄ in PBS for 1 hour, followed by a dehydration series with ethanol. The samples were then left in absolute ethanol for 30 minutes and placed onto glass coverslips, followed by critical point drying and coating with gold. Micrographs were recorded using a JEOL JSM-6700F FE-SEM scanning electron microscope operating at 10 kV. The obtained micrographs shown in FIG. 1I are representative of three independent experiments.

As shown therein, following overnight treatment, membrane fusing, clumping, and disintegration were abundant in the treated bacteria, which appeared deflated compared to the control bacteria, thus pointing to the bacterial membrane as a target of the tested nanostructures.

The effect of Fmoc-F5-Phe nanostructures on bacterial membrane permeation was also supported by a SYTOX Blue membrane permeation assay. SYTOX Blue is a cationic dye that cannot enter an intact cell unless its membrane is disrupted by external compounds. Inside the cell, SYTOX Blue stain binds to intracellular nucleic acids and fluoresces bright blue when excited with 405 nm violet laser light.

S. mutans bacteria were grown under anaerobic conditions in BHI medium (BD Difco) for 48 hours and diluted to 0.1 OD₆₀₀. Fmoc-F5-Phe or ultrapure water (100 μL) as control was added to 300 μL of bacteria and incubated for 3 hours in 37° C. The bacterial cells were centrifuged for 5 minutes at 3700 rpm and incubated with 1 μM SYTOX blue (Thermo Fisher Scientific) for 30 minutes at 37° C. The samples were washed three times in PBS and examined by confocal microscopy LSM 510, excited at 405 nm (Zeiss, Germany).

The obtained data is presented in FIG. 1J, and show that significant enhancement in the fluorescence of bacterial samples treated with Fmoc-F5-Phe nanostructures —of about 90%—was observed, as opposed to the control sample, in which less than 1% were stained with this dye. Taken together, these results demonstrate the substantial membrane disruption capabilities of the tested exemplary self-assembled nanostructures.

### Example 2

### Preparation and Mechanical and Optical Characterization of a Dental Composite Restorative

Composite antimicrobial dental materials were prepared by incorporation of antimicrobial peptide nanostructures as disclosed herein within dental resin composite restoratives. Nanostructures prepared as described herein were incorporated within a Filtek™ dental resin composite restorative (Filtek Ultima Flow resin composite restorative) while ensuring that the structures are efficiently and evenly distributed. This resin composite restorative was shown as not featuring antimicrobial activity [Matalon et al. Quintessence Int. 2009, 40, 327-332].

Nanostructures were added to the commercial pre-polymerized matrix at four different weight concentrations; 0.25, 0.5, 1 and 2%, by weight. Each sample was centrifuged for 1 minute at 3700 RPM and then mixed manually for three minutes followed by 1-minute centrifugation at 3700 RPM, and sonication for 5 minutes. Following sonication, samples were centrifuged for 1 minute, manually mixed for three minutes and then centrifuged for 1 minute at 3700 RPM. The resulting amalgamated restoratives were polymerized for 40 seconds per individual sample, by Elipar Trilight (3M, ESPE); a high-performance light polymerization unit to thereby obtain the polymerized dental resin composite restoratives. Even distribution was confirmed via EDX.

FIGS. 2A-B present optical images and exemplary EDX analyses of a polymerized dental composite restorative upon exposure to 40 seconds UV curing per individual sample, as described hereinabove, having the Fmoc-Pentafluoro-Phe peptide nanostructures (2% by weight) incorporated therewithin, compared to plain dental composite.

As shown, the incorporation process yielded a uniform and even distribution of the nanostructures within the amalgamated restorative composite.

The nanoscale size of the self-assembled structures is assumed to allow for their facile and functional incorporation into dental resin composites commonly used as clinical restorative materials.

The effect of the incorporation of peptide nanostructures in the polymerized dental resin composite restorative on the structural integrity of the resin composite restorative was tested. A Shear-Punch Test was carried out on samples incorporating varying weight ratios of Fmoc-Pentafluoro-Phe.

A Shear-Punch Strength Test was performed according to Mount et al., Aust. Dent. 1996; J 41: 116-23. Shear Punch Strength Test. Briefly, a composition comprising the nanostructures dispersed in a pre-polymerized resin composite restorative was placed in 0.8 mm thick wash holders and light-cured to form flat parallel surfaces evenly supported and restrained by the holder. The samples were then placed in an Instron device (model 4502) for punching under a crosshead speed of 0.5 mm per minute. The maximum force applied (Fmax) was calculated as the mean of 10 different samples for each w/w % concentration of the tested nanostructure. Statistical analysis was carried out via one-way analysis of variance and Dunnett's post hoc test.

The obtained data is presented in FIG. 3A and show that no statistically significant difference was found between concentrations of 0.25%, 0.5% and 1% of Fmoc-F5-Phe as compared to the control (0%) (p≥0.144), in terms of Fmax (the maximal force applied to break/punch the sample), and that 2% of Fmoc-F5-Phe was inferior to the control by 9% (p=0.011), a percentage similar to the limit of coefficient of variation of the SPS test (8%). These data show that the incorporation of peptide nanostructures does not have a substantial effect on the structural integrity of the dental resin composite restorative. The data obtained in these tests further show no substantial effect of incorporating of the peptide nanostructures on the stiffness of the tested specimen (not shown).

Diametral Tensile Strength (DTS) Test was further performed to verify the difference in tensile properties (tensile strength and stiffness of the specimens characterizing the elasticity of the materials) of the 2% amalgamated material compared to the control (0%), as follows.

Disks (6 mm in diameter, 3 mm in height) of either control resin composite restorative or 2% nanoassembly-incorporated resin composite restorative were prepared in a Teflon mold similar to the specimens used for the punch shear strength. Specimens were loaded up to failure. The linear slope during loading was calculated, indicating the stiffness of the specimen, and the DTS was calculated by:

DTS=2P/DTS=2PhrDt

where P is the load at failure (N), D is the specimen diameter (mm), and t is the specimen height (mm).

The specimens were loaded via the above-mentioned loading machine using the same crosshead speed. Statistical analysis was carried out via T-test.

The obtained data is shown in FIG. 3B. No statistically significant differences were found in either strength or stiffness (p>0.155).

The inherent stability of the amalgamated materials was also demonstrated via a high performance liquid chromatography-based nanostructures release evaluation, which was carried out over 72 hours in sterile salvia.

Restorative composites incorporating 4 mg of the Fmoc-F5-Phe nanostructures to a final w/w % of 2% were examined following 24, 48 and 72 hours of incubation at 37° C. in sterile saliva.

After 24 hours of incubation, the resin composite restoratives released 0.9520 micrograms, less than 0.024% of the initial amount of incorporated nanostructures. Following 48 hours incubation, there was a tenfold decrease in the percentage of incorporated molecules released to 0.1805 micrograms, below 0.0046%. Following 72 hours incubation, 0.1088 micrograms were released.

Occlusal Fissure Stability and Optical Property Analyses were also performed as follows.

Occlusal fissures were made via a diamond bur and then restored utilizing either the 2% nanostructure-containing restorative composites or a control (0%) restorative. The samples were contained for 30 days at 37° C. in sterile PBS. A Spectroshade Micro-MHT dental spectrophotometer normalized to the Vita classical color guide was then utilized to evaluate the color of the tested samples.

As shown in FIG. 3C, large occlusal fissure restorations performed with both the 2% nanostructures-containing composite restoratives and the control (0%) remained intact and stable following a 30-day incubation at 37° C. in sterile phosphate buffered saline (PBS).

The effect of nanoassembly incorporation on the optical properties of the dental restorative composite, an esthetically important feature for their clinical use, was evaluated utilizing a Spectroshade Micro-MHT dental spectrophotometer normalized to the Vita classical color guide. As shown in FIG. 3D, both the nanostructure-containing restorative composite and the control were spectroscopically identified to be of the same Vita shade.

Without being bound by any particular theory, it is assumed that the non-substantial effect of the incorporation of the self-assembled nanostructures on the optical and mechanical properties of a dental restorative composite can be attributed to the size of the self-assembled structures and the low loading dose required for their conferral of antibacterial activity to the resin composite restoratives.

### Example 3

### Antimicrobial Activity

The antimicrobial activity of the polymerized composite materials was evaluated by Direct-contact kinetic analysis and by minimum inhibitory concentration (MIC) analysis, as follows.

Direct-Contact Kinetic Analysis:

This analysis was carried out as described in Weiss et al. Endod. Dent. Traumatol. 12, 179-84 (1996), with slight modification.

S. mutans bacteria were grown in anaerobic conditions in BHI media for 48 hours and then diluted to an OD₆₀₀ of 0.6 in BHI. 100 of these samples were deposited onto inserts (concaved plastic surfaces designed to be suspended in the wells of 96 well-plates) coated on one side with each of the nano-assembly incorporated resin composite restoratives (four different W/W % samples of the resin composite material were evaluated at 0.25, 0.5, 1 and 2% concentrations of each nanostructure) and then incubated for one hour at 37° C. Following incubation 2250 of BHI was added to each well so that the inserts were submerged in the BHI and the plates were sealed to ensure anaerobic conditions. Kinetic growth inhibition was determined by optical density measurements (650 nm) using a Biotek Synergy HT microplate reader. Kinetic analysis results displayed and end point dose dependency analysis (FIGS. 4A-C) are representative of three independent experiments conducted in quadruplet.

As shown in FIGS. 4A-C, the samples containing 0.25-1% nanostructures were able to partially inhibit bacterial growth in a dose dependent manner while 2% Fmoc-F5-Phe nanostructures were able to cause substantial (over 95%) bacterial growth inhibition. Treatment with resin composite containing 2% H-F5-Phe did not significantly affect bacterial growth while treatment with resin composite containing 2% Fmoc-Phe inhibited bacterial growth by 60%. Treatment with 0.25-1% H-F5-Phe did not affect bacterial growth while treatment with 0.25-1% Fmoc-Phe inhibited growth in a dose-dependent manner.

Live/Dead viability analysis:

To directly assess bacterial viability, treated and control bacteria were subjected to Live/Dead viability analysis, using the Live/Dead backlight bacterial viability kit and accompanying instructions, as follows.

S. mutans bacteria were grown in anaerobic conditions in BHI media for 48 hours and then diluted to an OD₆₀₀ of 0.6 in BHI. 100 μl of these samples were deposited onto inserts coated on one side with each of the polymeric matrices and then incubated for one hour at 37 degrees. Following incubation 2250 BHI was added to each well and the plates were sealed to ensure anaerobic conditions. At each time point (initial incubation, 1 hour and 24 hours) samples were taken and washed with saline, incubated for 15 minutes in a solution containing Propidium iodide and Syto9 (L13152 LIVE/DEAD® BacLight™ Bacterial Viability Kit, Molecular Probes, OR, USA) and washed with saline again. Fluorescence emission was detected using an ECLIPSE E600 (Nikon, Japan).

The obtained data is presented in FIG. 5. Green fluorescence of the Syto9 probe indicates bacterial cells with an intact membrane, while red fluorescence of Propidium Iodide (PI) indicates dead bacterial cells. As shown therein, following one hour of treatment large scale bacterial death was observed in samples treated with composite incorporating Fmoc-Pentafluoro-Phe, compared to those that were treated with Filtek™ alone, and this effect persisted for 24 hours.

### Example 4

### Cytotoxicity

The effect of the incorporation of Fmoc-F5-Phe on the cytotoxicity of the dental composite restoratives was tested. This was carried out by evaluating the effect of each amalgamated composite restorative (containing self-assembled nanostructures as described herein) on the viability of two cell lines; HeLa and 3T3 fibroblasts, using MTT analysis, as follows.

The 3T3 fibroblast cells and HeLa cells grown in Dulbecco's Modified Eagle's Medium (DMEM) supplemented with 10% fetal calf serum (FBS) (Biological Industries, Israel) were sub-cultured (2×105 cells/mL) in 96-well tissue microplates (100 μl per well) and were allowed to adhere overnight at 37° C. Inserts (in quadruplet) coated on one side with each of the nano-assembly incorporated resin composite restoratives were placed into the wells containing the adhered cells. After incubation for 18 hours at 37° C., cell viability was evaluated using the 3-(4,5-dimethylthiazolyl-2)-2,5-diphenyltetrazolium bromide (MTT) assay. Briefly, 10 μL of 5 mg/mL MTT dissolved in PBS was added to each well. After 4 hours incubation at 37° C., 100 μl of the extraction buffer [20% SDS dissolved in a solution of 50% dimethylformamide and 50% DDW (pH 4.7)] were added to each well, and the plates were incubated again at 37° C. for 30 minutes. Finally, color intensity was measured using an ELISA reader at 570 nm. The results presented are the mean of three independent experiments conducted.

FIGS. 6A-D presents the obtained data, which revealed that the incorporation did not change the effect of the dental resin composite restoratives on eukaryotic cell viability.

These results indicate the enhanced antibacterial potency of the composite restorative material, as the cytotoxic activity is not directed toward mammalian cell lines but only toward bacterial cells. While several restorative and resin-based materials have been embedded with bioactive compounds, a high-dose loading of these compounds is usually required to achieve their antibacterial activity, resulting in low biocompatibility. The low dosage needed to achieve successful antibacterial activity by the self-assembled nanostructures described herein and the reduce cytotoxicity thereof toward mammalian cells, render the composite restorative materials described herein highly biocompatible.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition comprising a dental formulation and at least one self-assembled nanostructure incorporated in said dental formulation,
said nanostructure being formed of self-assembled plurality of aromatic molecules, wherein each of said aromatic molecules comprises an aromatic amino acid.

2. The composition of claim 1, wherein in at least a portion of said plurality of aromatic molecules, each of said aromatic molecules comprises an aromatic amino acid having an end-capping moiety attached thereto.

3. The composition of claim 2, wherein said end-capping moiety is an aromatic end-capping moiety.

4. The composition of claim 1, wherein in at least a portion of said plurality of aromatic molecules, each of said aromatic molecules comprises a peptide of from 2 to 6 amino acid residues, at least one of said amino acid residues being said aromatic amino acid.

5. The composition of claim 4, wherein said peptide is an end-capping modified peptide.

6. The composition of claim 5, wherein said end-capping modified peptide comprises an aromatic end-capping moiety.

7. The composition of claim 1, wherein said aromatic amino acid is phenylalanine.

8. The composition of claim 1, wherein in at least a portion of said aromatic molecules, said aromatic amino acid is a halogenated aromatic amino acid.

9. The composition of claim 8, wherein said halogenated aromatic amino acid is pentafluoro-phenylalanine.

10. The composition of claim 1, wherein said plurality of aromatic molecules comprises a plurality of Fmoc-pentafluoro-phenylalanine.

11. The composition of claim 1, wherein said plurality of aromatic molecules comprises a plurality of Fmoc-phenylalanine.

12. A method of treating or preventing a dental and/or periodontal infection, the method comprising contacting an infected area in the oral cavity of a subject in need thereof with the composition of claim 1.

13. A method of treating a dental, periodontal or orthodontic condition in which treating or preventing a bacterial infection and/or reducing, inhibiting or retarding biofilm formation is beneficial in a subject in need thereof, the method comprising contacting an organ or a tissue in the oral cavity of the subject with the dental composition of claim 1.

14. A composite material comprising a polymeric matrix usable in a dental, periodontal or orthodontic application and at least one self-assembled nanostructure incorporated in and/or on said polymeric matrix, the composite material being prepared upon subjecting the composition of claim 1 in which said dental formulation is a curable formulation to conditions for effecting curing of said curable formulation.

15. The composite material of claim 14, being a dental restorative material.

16. A composite material comprising a polymeric matrix usable in a dental application and at least one self-assembled nanostructure incorporated in and/or on said polymeric matrix, wherein:
said polymeric matrix is usable in a dental, periodontal or orthodontic application; and
said at least one nanostructure comprises a nanostructure formed of a plurality of aromatic molecules, each of said aromatic molecules comprising an aromatic amino acid.

17. The composite material of claim 16, wherein in at least a portion of said plurality of aromatic molecules, each of said aromatic molecules comprises an aromatic amino acid having an end-capping moiety attached thereto.

18. The composite material of claim 16, wherein said end-capping moiety is an aromatic end-capping moiety.

19. The composite material of claim 16, wherein in at least a portion of said plurality of aromatic molecules, each of said aromatic molecules comprises a peptide of from 2 to 6 amino acid residues, at least one of said amino acid residues being said aromatic amino acid.

20. The composite material of claim 19, wherein said peptide is an end-capping modified peptide.

21. The composite material of claim 16, wherein said aromatic amino acid is phenylalanine.

22. The composite material of claim 16, wherein in at least a portion of said aromatic molecules, said aromatic amino acid is a halogenated aromatic amino acid.

23. The composite material of claim 16, wherein said plurality of aromatic molecules comprises a plurality of Fmoc-pentafluoro-phenylalanine.

24. The composite material of claim 16, wherein said plurality of aromatic molecules comprises a plurality of Fmoc-phenylalanine.

25. The composite material of claim 16, being a dental restorative material.

26. A process of preparing the composition of claim 1, the process comprising:
mixing said at least one nanostructure and said polymeric precursor formulation, said mixing comprising repetitively subjecting a mixture of said at least one nanostructure and said polymeric precursor formulation to manual mixing, centrifugation and/or sonication.

27. A method of treating a dental, periodontal or orthodontic condition in which treating or preventing a bacterial infection and/or reducing, inhibiting or retarding biofilm formation is beneficial in a subject in need thereof, the method comprising contacting an organ or a tissue in the oral cavity of the subject the composite material of claim 16.

28. A composition comprising at least one nanostructure formed of self-assembled plurality of aromatic molecules, wherein each of said aromatic molecules comprises a halogenated aromatic amino acid, the composition being for use in inhibiting, reducing or retarding a formation of a bacterial load in and/or a substrate.

29. An article-of-manufacture comprising a polymeric matrix and the composition of claim 28 incorporated in and/or the polymeric matrix.

30. A method of inhibiting, reducing or retarding a formation of a bacterial load in and/or a substrate, the method comprising contacting the substrate with the composition of claim 28.
